(19) United States
(12) Patent Application Publication
(10) Pub. No.: US 2019/0125973 A1
(43) Pub. Date: May 2, 2019

(54) COMBINATION NEEDLELESS HYPODERMIC INJECTOR AND COVER SHEET AND RELATED METHODS

(71) Applicants: H.N.S. International, inc., Santa Ana, CA (US); University of Toyama, Toyama (JP)

(72) Inventors: Masayoshi Fukushima, Orange, CA (US); Makoto Nakamura, Toyama (JP); Satoshi Fukui, Toyama (JP)

(73) Assignees: H.N.S. International, inc., Santa Ana, CA (US); University of Toyama, Toyama (JP)

(21) Appl. No.: 16/091,510

(22) PCT Filed: Apr. 5, 2017
(86) PCT No.: PCT/US17/26188

§ 371 Date: Oct. 4, 2018

Related U.S. Application Data
(60) Provisional application No. 62/318,632, filed on Apr. 5, 2016.

(51) Int. Cl. A61M 5/30; A61K 31/728; A61F 13/02

(52) U.S. Cl. CPC A61M 5/30; A61M 2005/3022; A61F 13/02; A61K 31/728

(57) ABSTRACT

A combination needleless injector device for delivering a therapeutic effective amount of a fluid agent subcutaneously and a cover sheet for facilitating delivery of residual fluid not delivered by the needleless injector. The needleless injector has a discharge end and a drive end. The discharge end has a relatively small nozzle for fluid to discharge therethrough for delivery subcutaneously to a patient without a needle and the drive end has a piston held by a trigger and wherein a spring is pushed against the piston to drive the piston to then drive a plunger through the ampule to discharge the fluid medicament. The cover sheet has a liquid impermeable layer and an adhesive layer for surrounding an injection site.

### FIELD OF ART

Needleless hypodermic drug delivery systems and spring actuated jet injection devices utilizing a high pressure liquid stream to pass a medicament or other liquid through the skin, subcutaneously, are described. Cover sheets or dosage sheets are also described for use with the needle injectors, such as following the needleless injection to facilitate the delivery of residual fluids.

### BACKGROUND

Jet injection devices administer intramuscular and subcutaneous medications without the use of needles. Among the many advantages of jet injection are the reduction of apprehension associated with needles, the elimination of needle stick injuries, the reduction of environmental contamination associated with needle disposal, and possibly the reduction of pain. Jet injection devices are useful in a wide range of drug therapies including immunization vaccines, hormones and local anesthetics, as well as the administration of insulin to the diabetic population, where individuals may require a number of daily injections. Thus, their use has become of increasing interest, particularly by persons of limited physical ability such as the elderly, or the very young.

Injectable medications fall into two different categories; namely, unit dose drugs such as vaccines and analgesics and variable dose drugs such as insulin where the dose size must be adjusted to meet the immediate needs of the individual at the time of administration. When a variable dose is required, as in the case of the administration of insulin, a very accurate amount of medication must be transferred to a variable dose ampule. Insulin doses are typically marketed in 3 ml and 5 ml syringe cartridges, as well as provided in bulk in a standard 10 ml medication vial. These dose categories and differing medication source containers, therefore, impose conflicting design requirements on ampules or syringe compartments provided in prior art jet injection systems.

Theoretically, the expelled medicinal or therapeutic liquid from a needle-free or needleless injector is supposed to be injected and penetrate into the skin from the internal space between the keratinocytes, which cover the surface of skin. However, some small quantity of medicine can remain external to the skin, such as not enter the skin, during the injection process. Although such residual quantity of drug should be delivered subcutaneously, it is typically abandoned, such as wiped clean with a cloth.

### SUMMARY

Needleless injector devices and cover layers for covering an injection site following a needleless injection are disclosed. The needless injector can comprise an ampule with a discharge end having at least one discharge nozzle and a drive end comprising a spring and a piston for pushing against a plunger slidably located inside the ampule.

Following a needleless injection, residual fluid not delivered by the needleless injector device can be transferred through the skin using a cover sheet, which can have a liquid impermeable layer and an adhesive ring.

Aspects of the present disclosure include a needleless injector device or needleless injector assembly. Broadly speaking, the needleless injector device can comprise a drive end component, or drive end for short, and a discharge end component, or discharge end for short. The drive end, also sometimes refers to as an injection driver, injector end, spring end or simply injector, can comprise an elongated body or housing comprising a trigger for holding back and subsequently releasing a motive force located inside the elongated body when discharged to propel a piston, also located inside the elongated body, to then propel a plunger located inside the discharge end, as further discussed below.

In one example, the drive end comprises a safety mechanism or safety lock that is displaceable to allow the trigger to be activated. As shown, the safety lock can be a ring that is slidable about the elongated body to unlock the trigger, such as to provide space for the trigger to be depressed. In other embodiments, the safety lock is rotatable or pivotable to unlock the trigger, such as to provide space for the trigger to move, pivot or rotate. In still yet other examples, the safety lock is both slidable and rotatable to unlock the trigger. In other examples, the safety feature comprises a frangible tab that is removable to provide space for the trigger to be depressed or released.

The discharge end is configured to hold a volume of fluid, such as a fluid medicament or a quantity of therapeutically effective ingredient, vaccine, flu shot, insulin, local anesthesia, lidocaine, hyaluronic acid, tetanus shot, etc., for subcutaneous delivery of a patient. For example, the discharge end can contain a clinically effective amount of hyaluronic acid for injecting the facial area for cosmetic treatment. As shown, the discharge end is an ampule comprising a discharge head having a discharge tip and a discharge base having a flange and a coupling end, which in the present embodiment can comprise a threaded end.

The discharge head can have a relatively larger in cross-sectional dimension than the body of the ampule. In other examples, the two are the same or the body can be larger. In other examples, the coupling end is a quick release end without threads. The discharge end is configured to threadedly engage with or to the drive end.

In other examples, the ampule has a quick release cam surface for engaging a mating surface on the discharge end. The discharge end, for example an ampule, can have an elongated body of a size and dimension for sufficiently holding a desired volume of fluid medicament for subcutaneous delivery to a patient. In some examples, the assembly, such as the ampule, is sized for use in dental applications, such as for delivering local anesthesia to the gum or mouth.

In one example, the discharge end is made from a cyclo-olefin-copolymer (COC) material, such as from TOPAS and APEL Mitsui Chemical of Japan. It is believed that fluid medicament may be stored in the ampule made from COC for a much longer period than for ampules made from other thermoplastic or engineered plastic materials. This allows for the ampules to be pre-filled and stored with different fluid medicaments and refrigerated so that they may be readily available for use with drive ends of the present disclosure. In still other examples, the discharge end 14 is made from a plastic material, such as a thermoplastic material, selected to have impact resistant characteristics. For example, the discharge end may be made from plastic injection molding using an acrylic-based polymer, such as ACRYLITE® and HYGARD®, the latter being made from a multi-layer of polycarbonate and acrylic. Optionally, the discharge end may be made from plastic injection molding using a polycarbonate (PC)-based material, such as LEXAN®, MARKOLON®, SAFEGUARD®, and SAFEGUARD HARDCOAT®. In still other examples, the discharge end may be made from plastic injection molding using a polyethylene (PE)-based material, such as POLYSTONE® PG100, POLYSTONE® 500, and POLYSTONE® MATROX.

The drive end having the spring motive force is preferably made from a hard plastic, such as high density polyethylene (HDPE), polycarbonate (PC), polyvinyl chloride (PVC), COC or other comparable hard plastic. In one example, the elongated body is made from two separate housing halves, such as by plastic injection molding two different opaque sections, that are joined together along a lengthwise seam by welding, gluing, detents, or combinations thereof. In other examples, the two sections can have a seam formed orthogonal or at an angle to the lengthwise axis.

The elongated body may comprise a plurality of ribs, such as elongated ribs, that extend at least partially along the length of the elongated body. In other examples, the body has a smooth outer surface contour, a plurality of bumps or projections, or combinations thereof. A pair of mounting flanges are provided near the distal end of the drive end with each comprising a cradle for receiving or accommodating a pivot pin or shaft. The opening of each cradle can be sized and shaped to receive the pivot pin or shaft in a snap fit arrangement.

The pivot shaft is operatively connected to the trigger so that when the trigger is pushed, it rotates about the pivot shaft. In some examples, the trigger and the pivot shaft are unitarily formed, such as by injection molding, co-molding, or insert molding. The trigger can include a plurality of exterior gripping features, which can be ribs, projections, or bumps formed on the outer surface to facilitate gripping. In other examples, a pin is connected or mounted with the body of the discharge end and the trigger is equipped with a pair of cradles for snapping onto the pin mounted to the injector body.

The drive end can comprise a distal opening comprising a threaded bore for receiving the threaded end of the discharge end, such as the ampule. A rail or track can be provided at the distal end of the drive end for accommodating a channel formed in the interior bore of the safety lock so that the channel can ride along the rail or track and is also used for rotational alignment.

The track can ensure that the ring is rotationally aligned so that a protrusion or raised bump formed on the exterior surface of the safety ring aligns with the trigger so as to provide a physical presence under the trigger to prevent the trigger from triggering until the obstruction is removed. In other examples, the raised bump can be formed on the safety ring can rotatable from a position away from the trigger to a position below or under the trigger to provide the physical barrier for preventing triggering. In yet other examples, the trigger can be provided with a collapsible or frangible leg that severs or collapses upon exertion of a sufficient downward pressure on the trigger.

An end cap can be provided at the proximal end of the drive end. The end cap can be provided to cover or close-off the proximal opening of the elongated body after installation of various injector components, such as a spring and/or a piston. In one example, the end cap can be threadedly engaged to threads located on the elongated body. In another embodiment, as further discussed below, the end cap can be provided with a slidable mechanism for engaging corresponding features located at the proximal end of the elongated body to close the proximal opening of the elongated body. For example, the end cap can comprise an end wall and a rim having an open passage through the rim so that the cap can slide over the proximal opening via the passage through the rim.

Flanges can be formed on the rim of the cap to then engage tracks or channels on the elongated body to secure that the cap is attached thereto and prevent from displacing in the axial by the force of the spring. Said differently, physical restraints can be employed between the elongated body and the cap, such as sliding rails, tongue-groove, etc., to ensure engagement. A detent, such as a pin and a boss, may be used to then secure the cap to the elongated body from being displaced radially relative to the lengthwise axis of the body after the initial engagement. The slidable mechanism between the cap and the elongated body can permit easy subsequent removal of the end cap from the elongated body to expose the proximal opening to facilitate optional removal of the spring located inside the elongated body.

The ampule can include an enlarged discharge head, which can be larger in outside diameter than the outside diameter of the elongated body. The enlarged discharge head can have a generally constant outside diameter along a length of about 10% to about 35% of the total length of the ampule. Optionally, the wall thickness of the elongated body can be constant and the enlarged discharge head can be omitted. Exteriorly, the discharge head can comprise a plurality of generally parallel fins to facilitate gripping when mounting the discharge end onto the drive end.

At least one outlet nozzle can be provided at the discharge tip of the ampule. The outlet nozzle, which can have a diameter or bore size in the range of four thousandths to twelve thousandths, is sized and shaped to allow fluid inside the ampule to be discharged therethough to subcutaneously deliver an injection of fluid to a patient. To facilitate discharging fluid out the nozzle of the ampule, a plunger can be slidably provided in the interior cavity of the ampule.

When the plunger is advanced, such as when pushed by a spring or when pushed by a piston pushed by a spring following activation of the trigger, the plunger tip pushes fluids inside the ampule out the discharge nozzle at the discharge tip. The plunger can have a plunger tip for dynamically sealing against the interior surface of the ampule to discharge fluid out the nozzle, similar to a plunger inside a barrel of a syringe.

Further aspects of prior art needleless injectors are disclosed in U.S. Pat. Nos. 6,558,348; 5,704,911; 5,569,189; and U.S. Pat. No. 5,499,972. To the extent structures and features disclosed in these four prior art patents do not conflict with expressly disclosed features of the present disclosure, they are expressly incorporated herein by reference for their teachings.

A helical spring and a piston can be positioned inside the interior cavity or bore of the elongated body of the drive end. The piston can have a piston head and a piston stem defining a shoulder therebetween.

Both the piston head and the piston stem can be annular or round in nature, along an end cross-section. The length of the stem and the length or thickness of the piston head can vary. The spring, which can be made from a metal material, such as from carbon steel, can be placed in a compressed state with the piston moved proximally of the latch pin located on the trigger, which abuts the piston face to hold the piston which then holds the spring in compression.

The spring of the present disclosure may be compressed or set to compress in the manner shown and described in the '911, '189 and '972 patents in the compressed or loaded position, such as by using a setting tool to push against the piston head to compress the spring and allowing the latch pin to move distal of the piston face to retain the spring in the compressed or loaded position. The latch pin can be a projection formed integrally or unitarily with the trigger. In another example, the latch pin can be separately formed and subsequently attached to the trigger. The latch pin can have a generally flat or planar surface on the proximal side for abutting the piston face.

The compressed spring can exert a high spring force against both the shoulder on the piston and the interior surface of the end cap. As the end cap can pivot due to a gap or slack in the mounting channel with the elongated body, as further discussed below, the spring force can cause a secure pin located at the proximal end of the body and a secure boss, similar to a bore or a recess, located on the end cap to engage. In other words, the spring can force the cap to pivot relative to the proximal end of the elongated body, which can then cause the secure pin to engage the secure boss to ensure retention of the cap to the body.

The engagement between the secure pin and the secure boss can prevent the cap from sliding radially to separate from the engagement between the flanges on the cap and the channels on the elongated body. This feature allows the end cap to latch onto the elongated body and held at the proximal end of the elongated body without threads. In other examples, the cap can have two channels and the elongated body can have two flanges that engage the channels to secure the cap to the body.

The spring force acting on the end cap can provide added resistance against potential unlatching between the secure pin and the boss. For example, the spring force can cause the pin to engage the secure boss to prevent sliding the cap radially relative to the lengthwise axis of the body until the spring force is reduced or removed. In other examples, the cap can have a pin and the elongated body can have a secure boss. In still other examples, the cap can be threaded to the body and held there by the threaded engagement with further securement provided by the force of the spring acting on the interior surface of the cap.

In practice, a plunger can be slidably positioned inside the ampule and the piston inside the drive end is configured to push a proximal end of the plunger when the trigger is depressed to release the spring, which then forces the piston against the proximal end of the plunger to propel the plunger in the distal direction to expel fluid inside the ampule out the one or more nozzles at the discharge end of the ampule. The plunger can have a plunger tip, similar to a plunger on a syringe, to dynamically seal against the interior surface of the ampule.

The safety lock can be moved forward or distally or rotated to move the exterior protrusion on the safety lock away from the trigger and provide clearance between the trigger and the exterior surface of the body for triggering. In other words, the safety lock can be moved away from the trigger to provide clearance for the trigger to move for triggering or releasing the spring.

When the trigger depressed at the push end to release or move the latch pin away from the piston face, such as to move the latch pin radially away from the lengthwise axis of the elongate body, the spring can be released to then propel the plunger to discharge fluids located inside the ampule. The push end of the trigger can be arranged to face or point in the distal direction, i.e., points towards the ampule. This pushing of the trigger at the push end can release the spring and allow the spring to rapidly expand to propel the piston into the plunger to then propel the plunger into the interior of the discharge end component to expel fluid out the nozzle at the discharge tip of the discharge end or ampule. Consequently, an injection can be made by placing the discharge tip of the injection assembly against the skin of a patient, pulling or depressing the trigger to release the spring to then push the piston against the plunger and discharging fluid medicament held inside the hollow cylinder of the discharge end into the patient.

When the spring expands, such as after fluid discharge out the one or more nozzles on the ampule, it exerts a lower spring force on the interior surface of the end cap than when the spring is in the compressed state. Consequently, less force is exerted on the end cap by the spring after the spring is released. Because of the lower force, the cap located at the proximal end of the drive end is less torqued or slanted about its upper end. This less slanted position may further be facilitated by pushing on the cap near the upper end. The manner in which the cap engages the body using a slidable mechanism is further discussed below. Thus, he cap can be positioned generally square or vertical relative to the end edge of the body when the spring is released.

In contrast, when the spring is compressed and ready to be released by depressing the trigger, the spring force exerts a greater outward force on the end cap and causes the end cap to slant to force the secure pin and the secure boss to engage. In an example, the slanting can be caused by holding the end cap tight at one end and loose at another end so that the end that is loosely held can move to the constraint of the holding. For example, tracks can be used with tapered gaps to hold the cap tight at the end with a small gap and loose at the end with a larger gap.

The lower load on the end cap, after the spring expands, allows a user to manipulate the cap to separate the cap from the elongated body of the drive end. For example, when the secure pin and the secure boss are not engaged, the end cap can readily slide radially to separate the cap from the elongated body. Removal of the end cap allows the spring to be removed from the elongated body through the proximal opening of the elongated body. This then allows a user to separate a metal component, i.e., such as the spring, from the various thermoplastic components. The components of the needleless injector, after the spring is removed, are all or are mostly non-metallic and therefore can easily be placed into a recycling bin for recycling. The separated spring may be re-used, if desired, otherwise discarded in an appropriate bin for disposal or recycling.

In one example, the end cap may be pushed in the distal direction near the upper end of the cap with a finger or a thumb to further facilitate separating the secure pin from the secure boss. For example, using a finger and pushing the cap near an end of the cap that is loosely held back against the elongated body can reduce the slanting of the cap relative to the body to separate the secure pin from the retaining boss. The ability of the cap to cant or slant is provided by an engagement between a flange inside a tapered channel. For example, the cap can have a flange and the elongated body can have a tapered channel. The flange can be held loosely at one end of the tapered channel and more tightly at the other end of the tapered channel. In some examples, the cap can have a tapered channel and the elongated body of the drive end can have a flange.

The tapered channel, which can be wider at one end than at another end, provides room for the flange to move within the channel. Once the secure pin and the secure boss are clear or spaced from one another, the end cap can slide radially relative to the longitudinal axis of the device to separate from the housing. In an alternative embodiment, the secure pin may be located on the end cap and the secure boss may be located on the elongated body. As shown, a plurality of spaced apart projections or protrusions may be incorporated at the rear surface of the end cap to provide added traction for the finger or thumb when sliding and removing the end cap.

The end cap can incorporate two engagement flanges for mating engagement with respective channels formed at the proximal end of the elongated body. The flanges can be formed on the rim of the cap. In one example, the channels can each taper by having a relatively large width at an end or edge of the elongated body and tapers inwardly as it proceeds downwardly towards the other edge of the injector body.

If the parting line of the two-part elongated body of the drive end defines a vertical plane running lengthwise with the injector assembly, one channel is located on an outer surface of the body on each side of the vertical plane. The cap can have a corresponding flange located on the inside or interior of the cap for engaging the exteriorly located channels. The tapered channel narrows as it extends towards the terminal end or closed end of the channel. When the cap is placed over the proximal end of the elongated body, the two internal flanges on the cap engage the two outer channels on the elongated body. This can prevent the cap from being displaced axially in the proximal direction.

When the two engagement flanges are fully engaged to the two channels, the end cap closes off the proximal opening of the elongated body and the secure boss and secure pin cooperate to prevent the end cap from being displaced from the elongated body, in addition to the spring force acting on the end cap to bias the pin and the secure boss to engage. The tapered channels can provide a degree of freedom by allowing the engagement flanges on the cap to move within the confines of the channels. This is especially true after the spring has released and a lower spring force is acting on the end cap.

After the end cap is removed from the elongated body, a user can easily remove the spring and safely discard it, such by pulling the spring out of the elongated body with a hand or rotating the device and pointing the proximal end down so that the spring drops out of the proximal opening under its own weight. Thus, the end cap allows for an environmental friendly device that facilitates separating metallic components from non-metallic components for recycling. In other examples, the end cap is threaded onto the elongated body.

The piston can include features to prevent it from coming out the proximal opening when the spring is being removed. In one example, the piston is incorporated with a notch on the enlarged drum or head to prevent it from moving proximally of a plate or flange near the proximal opening. Other means for preventing the piston from being displaced out of the proximal opening may be used without deviating from the spirit of the present disclosure.

From the present disclosure, the present devices, systems, and methods are understood to include a needleless injector comprising an ampule and a plunger located in an interior cavity thereof connected to an injector driver comprising an elongated body, a piston, a trigger, a compression spring, and an end cap; wherein the end cap is pivotable or cant-able from a more vertical position to a more slanted position relative to the lengthwise axis of the elongated body. For example, the cap may define a plane that is at about 65 to 85 degrees from perpendicular with the lengthwise axis of the elongated body when the spring is held compressed. The cap can then slant less from about 80 degrees to a generally vertical position at about 90 degrees with the lengthwise axis of the elongated body when the spring is no longer held compressed by any part of the trigger, whether directly or indirectly, such as when the spring expands following an injection.

In a particular embodiment, a secure pin and a secure boss engage one another at the proximal end of the elongated body to secure the end cap to the elongated body. The end cap being removable from the elongated body by separating the secure pin from the secure boss. In one example, after the secure pin is separated from the secure boss, the end cap can slide radially relative to longitudinal length or axis of the elongated body to separate the end cap from the elongated body. This allows the user to readily remove various injector components from the elongated body to then recycle, re-use, and/or dispose.

The present disclosure is further understood to include an all elastic and/or thermoplastic needleless injector, other than for the spring, which can be made from a metal. Thus, upon removal of the spring from the elongated body, the needleless injector may be placed inside a recycling bin for non-metallic materials to be recycled. Features of the present disclosure are therefore understood to include an environmentally friendly device that is readily capable of recycling by allowing easy access to the components of the assembly to separate metallic from plastic components. In an example, this is facilitate by incorporating a cap with easy installation and removable to facilitate separation of the cap from the injector body for removal of metallic components from non-metallic components.

A cover sheet can be used following an injection with any of the disclosed needleless injector device. In an example, the cover sheet can be made from a single ply or a multi-laminate layer. The cover sheet can comprise a fluid or liquid impermeable main layer used as a backing and comprising an upper or outer surface and a lower or inner surface that faces the skin when the cover sheet is applied onto the skin, as further discussed below. The main layer has a perimeter and an inner surface edge adjacent the perimeter for use with an adhesive layer, such as a pressure sensitive adhesive. A removable release layer is provided to protect the adhesive layer until use, which should be made from or is provided with an easily removable surface to readily separate from the adhesive layer when using the cover sheet. The cover sheet can have an overall size or diameter to fit any injection site, ranging from about 0.5 inch in diameter and up. In an example, the cover sheet is individually wrapped in a moisture and liquid impermeable over-wrap. A container or package of cover sheets can comprise a plurality of individually wrapped cover sheets, similar to a first-aid bandage package.

In an example, the impermeable layer is made from a pliable thermoplastic or polymer material that can flex to follow the shapes or contours of the skin. Examples of thermoplastics and polymers useful for the backing layer are polyolefin, polyester, polyethylene, polyethylene vinyl acetate block copolymers, polyurethane, polyvinyl alcohol, polyvinylidene, polyvinylidene chloride, polyamide, ethylene-vinylacetate copolymer, ethylene-ethylacrylate copolymer, and polypropylene. The backing layer can also comprise laminates of one or more of the foregoing polymers. In an example, the liquid impermeable layer has a thickness of about one thousandths to about four thousandths of an inch. In other examples, the thickness range can be greater, such as up to ten thousandths or greater.

In an example, the liquid impermeable layer of the cover sheet has adhesive applied to the inner surface edge adjacent the perimeter, but not to the central portion of the inner surface. In other words, the inner surface has a surface area without any adhesive and is surrounded by a ring of adhesive. Thus, the adhesive ring can define a region external of the adhesive ring without adhesive and a region internal of the adhesive ring without adhesive. In some examples, the adhesive layer can be applied to all or substantially all of the inner surface of the inner surface side of the liquid impermeable layer.

The adhesive layer can form a ring around a central portion of the inner surface of the liquid impermeable layer. The ring can embody any number of shapes. For example, the shapes can be round, oval, square, polygonal, irregular, etc. The adhesive ring can have an enclosed or continuous perimeter or band. In an example, the ring of adhesive generally matches the shape of the cover sheet. In other examples, the shape of the cover sheet and the ring of adhesive can have shapes that differ from one another. For example, the impermeable layer can have a square or rectangular shape while the ring of adhesive can have an oval or circular shape.

When the cover sheet is applied over an injection site, such as following an injection using a needleless injector of the present disclosure, the cover sheet, with the release layer removed and the inner surface facing the skin, can be applied onto the skin so that the adhesive ring wraps or circles around the injection site and the injection site is retained within the inside perimeter of the adhesive ring. Residual medicinal fluid not delivered subcutaneously by the needleless injector can be captured under the cover sheet and within the adhesive ring to be deposited transdermally by the cover sheet through the puncture formed by the needleless injector.

Thus, aspects of the present disclosure is understood to include a combination needleless injector and cover sheet usable for delivering a therapeutic amount of liquid subcutaneously and residual therapeutic fluid not delivered by the needleless injector device through the skin after formation or production of at least one micro-pore or microstructure, such as a micro-puncture or small puncture formed by the needleless injector.

The adhesive can be a pressure sensitive adhesive, which is understood to be a viscoelastic material that remains tacky and that can adhere to the skin with the application of pressure, from a very slight or light pressure to any higher pressure. Typically, the adhesive can be made from a polymer based material mixed with plasticizers, tackifiers or other additives. Suitable pressure sensitive adhesive includes the polyacrylate adhesives and acrylic adhesives.

An alternative cover sheet can comprise an outer impermeable layer similar to that of FIG. 10, an inner layer, an adhesive layer similar to that of FIG. 10, and a release layer similar to that of FIG. 10. In an example, the inner layer can comprise an agent carrying layer. For example, the agent carrying layer can be an inner adhesive layer comprising a therapeutically effective amount of drug. Suitable transdermal drug delivery devices include gelled or liquid reservoirs, such as in U.S. Pat. No. 4,834,979; devices containing matrix reservoirs attached to the skin by an adjacent adhesive layer, such as in U.S. Pat. No. 6,004,578 (Lee, et al.); and devices containing pressure-sensitive adhesive reservoirs, such as in U.S. Pat. Nos. 6,365,178, 6,024,976, and 6,149,935. These devices are known as “drug-in-adhesive” patches and the disclosures of each of which are expressly incorporated herein by reference. In some examples, the drug carried by the inner layer is hyaluronic acid. In other examples, the inner layer can embody an alginate dressing.

Following an injection using a needleless injector of the present disclosure, the drug permeability of the skin at the injected area has been modified or induced by the high pressure stream expelled from the needle-free device. Thus, any residual drug that has not entered the skin can readily absorb at the injection site. The cover sheet is configured to be placed over the injection site to form a boundary layer around any residual medicine and to facilitate absorption of the remaining or residual therapeutically effective fluid on the exterior of the skin to enter the skin. The ring of adhesive can help secure the cover sheet to the skin and surround the residual therapeutically active ingredient.

In some examples, the injection site can comprise more than one passage or hole created by the needleless injector. For example, the ampule on the needless injector can have more than one discharge nozzles, such as two or more discharge nozzles to create two or more micro-pores or micro-structures. In other examples, two different injections can be performed using two different needleless injectors each with an ampule with only one discharge nozzle. In still other instances, the same drive end can be reset for use with two different ampules to perform two different needleless injections. The cover sheet of the present disclosure can then be placed over the injection sites to surround two or more micro-pores or structures after the injection(s) to facilitate absorption of any residual liquid or medicine located above the skin to enter the skin through the micro-pores or micro-structures formed by one or more needless injectors of the present disclosure.

After the injection, according to the rise of the permeability, the barrier function of the skin can be expected to decrease against the infectious agents, such as bacteria of the skin. Thus, the sterilized over-lapping sheet or cover sheet of the present disclosure can protect from such risk during the skin recovery.

The over-lapping sheet or cover sheet should be easily or readily removable from the skin after about 24 hours. In some examples, the cover sheet can be removed much sooner than 24 hours, such as 30 minutes, 45 minutes, or 1 hour after application. Other elapsed times are contemplated.

An alternative needleless injector device can include a discharge end and a drive end with quick connect/disconnect or a connection that is not a typical threaded connection. The engagement mechanism may be understood as a quick connect engagement mechanism to enable quick mounting of the discharge end onto the drive end.

In an example, the discharge end can comprise a pair of engagement wings for mating engagement with the pair of corresponding mating wings on the drive end. The engagement wings on the discharge end each can comprise a ramp for sliding engagement with a tapered fin formed on the corresponding mating wings of the drive end. The ramp and the tapered fin can act like a cam and axially load the two surfaces when the proximal end of the discharge end is placed into the drive end and rotated to engage the engagement wings with the mating wings. Other alternatives may be used to engage the discharge end with the drive end. For example, notches and/or other matching features may be incorporated on the discharge end and the drive end to ensure compatibility before they can be connected. Spring loaded quick disconnect may alternatively be used to connect an ampule to a drive end.

The present disclosure is understood to include a discharge end, such as an ampule, comprising a quick connect engagement mechanism. The present disclosure is further understood to include a pair of spaced apart engagement wings disposed about a proximal opening of the ampule for engaging with a pair of mating wings. In another embodiment, three spaced apart engagement wings are provided, which are evenly spaced about the proximal opening of the ampule. In another example, an injector end comprising a mating engagement mechanism is configured to quickly engage the engagement mechanism of the discharge end. In a particular example, two mating wings are provided at a distal end of the elongated body of the drive end for engaging spaced apart engagement wings on the discharge end. Other quick connect engagement mechanisms are contemplated, such as threads, detents, tongue and groove combination, etc.

In another example, the drive end, which may also be referred to as an injection driver, injector, power end, or spring end, can comprise a multi-part housing, a motive force device, such as a metallic helical coil spring, a piston, and an end cap for closing the proximal end of the drive end. When assembled, internal engagement flanges on the end cap engages the external channel on each of the two housing halves. The two housing halves after placing the motive force device and the piston therebetween, may be glued, welded, latched, or a combination thereof together.

A piston stopper can be formed near the distal end of the housing. The piston stopper can define an annular bore to permit cocking of the piston, such as by allowing a cocking tool to reach through the annular bore to compress the motive force device. After the motive force device is compressed, a trigger finger on the trigger can be positioned distally of the piston head to hold the motive force device in the compresses position, such as holding the distal face of the piston head.

A connection mechanism can be formed at the distal end of the drive end. The connection mechanism can comprise spaced apart latching tines each with internal gripping ridges or fingers for gripping a discharge end component, such as an ampule. The fingers can be located interiorly on the latching tines and ramped projections can be located exteriorly to enable a locking ring to ride thereover to close the tines over the discharge end component, such as an ampule.

The tines can be molded with an outward bias so that a locking ring is needed to force the tines inward to close against the discharge end component. A gap can be provided between two adjacent tines to receive flanged sections of the discharge end component of ampule for alignment purposes. In other embodiments, the channels can be reduced in size or gap or omitted as the ampule can be provided as a round structure to eliminate alignment issue.

The drive end can be assembled together and the seam either welded or glued. The end cap can now slide over the proximal end with the engagement flanges sliding into the channels located on either side of the housing at the proximal end. The drive end can now receive a trigger and a closing ring.

A trigger can comprise a trigger finger for projecting into the trigger hold slot to hold the piston and the motive force device in the cocked or ready to use positioned. The trigger can be assembled to the cradle by pushing the two pivot pins on either side of the trigger into the slotted receptacles of the two cradles, one on each housing half. The relative dimensions of the slotted receptacles can hold the pins in place after they are pushed therein.

A rocker spring can be provided between the trigger and the housing to pivot the trigger about the two pivot pins. In on example, the rocker spring may be omitted. In yet other examples, a compressible or elastic material, a leaf spring, or a V-spring may be used.

A closing ring can be provided for closing the tines. The closing ring may be placed over the tines by temporarily biasing the tines inwardly towards a longitudinal axis of the housing so that the opening of the closing ring can fit over and slide onto the housing. The closing ring can further incorporate a trigger lock that sits under the lock landing on the trigger to provide a physical barrier against rocking or pivoting by the trigger, which prohibits the trigger from pivoting about the pivot pins. The closing ring and the connection mechanism can further incorporate notches and detents for alignment purposes and for thwarting the closing ring from being displaced off of the connection mechanism.

In one example, the housing of the drive end may be assembled and then the piston and the spring slid into the housing via the proximal opening at the proximal end of the housing. The end cap is thereafter placed into engagement with the housing to close off the proximal opening. As discussed above, after an injection, the end cap may be removed to enable separation of the metallic spring from the other components of the injector end. This can allow the drive end and the discharge end component, such as an ampule, to be recycled without any metallic parts. Optionally, the trigger may be removed from the housing to enable removable of the rocker spring, if incorporated. If the rocker spring is not incorporated, the drive end may be recycled after the drive spring is removed through the proximal opening.

In an example, the ampule may be made from a cyclo-olefin-copolymer material (COC). The ampule can include a discharge end comprising a discharge nozzle and a mounting end comprising a mounting flange and a plurality of flange projections. The flange projections can be sized and shaped to slide between the gaps located between a plurality of tines of the connection mechanism. The flange can be sized and shaped to sit within the gripping ridges formed interiorly of the tines. Distal movement of the closing ring once the mounting end of the ampule is placed into the connection mechanism can force the tines to close down on the flange and secure the ampule to the drive end.

When the closing ring is moved distally out from under the trigger and the trigger is pressed at the triggering end, fluids inside the ampule can be expelled out the discharge end of the ampule under extremely high pressure. The assembly can therefore deliver a dosage subcutaneously without a needle.

An optional outer sleeve or outer layer can be provided with an ampule. In one example, a discharge end sleeve may be mounted over the discharge end of the ampule but not the entire length of the ampule. The discharge end sleeve may be made from a transparent or semi-translucent elastomeric or rubber material and placed over the discharge end to cushion the contact between the ampule and the recipient of the fluid to be delivered. The discharge and sleeve has an end wall and a skirt section. A distal opening is provided on the end wall of the discharge end sleeve to provide the needed opening for the discharge nozzle on the ampule. In other examples, the outer sleeve can extend the entire length of the ampule. In still yet other examples, the sleeve can extend beyond the connection end of the ampule to capture part of the drive end. This elongated configuration of the outer sleeve can further secure the connection between the ampule and the drive end and prevent incidental separation during use.

A body sleeve can be provided comprising an elongated body section for placement over the body of the ampule. The body sleeve, like the discharge end sleeve, can be made from a transparent or semi-translucent elastomeric or rubber material to enable viewing the inside fluid holding space of the ampule, such as to visually ascertain the medicinal level inside the ampule. The body sleeve can have two open ends.

In one example, both the discharge end sleeve and the body sleeve can be placed over the ampule. The combination discharge end sleeve and the body sleeve may be referred to as an outer layer. For example, the discharge end component may be made from a rigid plastic material and having an outer layer placed thereover or thereon. The outer layer can be made from a different material than the material used to make the ampule or the discharge end component.

In some embodiments, the outer layer is made or formed from two separate pieces while in other embodiments the outer layer is made from a single piece or unitarily formed as a single component. In still other examples, the outer sleeve can be made from more than two pieces or components. The body sleeve of the outer layer should extend more than half the length of the ampule.

In a particular example, the length of the body sleeve should extend to the mounting flange, such as contact with or nearly contact with the mounting flange. In other examples, the length of the outer layer can extend to around the half-way point of the length of the ampule and up to the mounting flange. If there is no mounting flange, then up to about the interface of the ampule and the injector end. It is believed that the impulse of force from the initial release of the motive force or spring and subsequent rapid movement of the plunger into the ampule can be dampened by the use of the outer layer. Thus, the outer layer should have a tight formfitting configuration around the body of the ampule to dampen some of the initial shock experienced by the ampule upon releasing the spring.

A one-piece sleeve can be thought of as a combination of the discharge end sleeve and the body sleeve. The sleeve has an end wall and an elongated body section. The sleeve is preferably sized and shaped to firmly wrap over the outside surface of the ampule but not so rigid or tight so as to deter or make assembling or mounting the sleeve over the ampule difficult. In some examples, the outer layer only has an elongated body section without a distal wall, such as being an open cylinder.

In some examples, a shrink wrap material can be used as an outer sleeve. In an example, a shrink wrap bag is provided for mounting over an ampule. The shrink wrap bag can have an elongated body section with a length, an inside diameter, and a distal opening formed on a distal end, distal wall, or end wall of the bag. The inside diameter of the shrink wrap bag can be sized sufficiently larger than the outside diameter of an ampule for which the outer layer is to be used with and the distal opening can have a perimeter that is sufficiently larger than the nozzle on the ampule so as not to obstruct fluid flow passing through the nozzle. In use, the shrink wrap bag may be slid over the body of an ampule and then subjected to heat so that the outer layer of the bag shrinks to form a tight fit around the ampule. Heat may be provided from electricity or from a gas source and may be part of a heated tunnel or an oven. Heated lamps may also optionally be used to shrink the outer layer.

In some examples, the shrink wrap bag may be made from a polymer material. Preferred polymers used for the shrink wrap bag include polyolefin, polyvinyl chloride (PVC), and polyethylene. The materials may be cross-linked or non-cross-linked. The shrink wrap bag may be formed to shrink in one direction (i.e., unidirectional or mono-directional) or in two directions (bidirectional). For example, the shrink wrap bag may be configured to only shrink in diameter but not length. In other examples, the shrink wrap bag may be configured to shrink both in diameter and along its length. The final length should extend at least to a half-way point of the length of the ampule and up to the mounting flange on the ampule, if any. If there is no mounting flange, then up to about the interface of the ampule and the injector end.

In an alternative embodiment, the outer layer comprises a shrink wrap open cylindrical section and a discharge end sleeve, similar to the end sleeve of FIG. 21. The open cylindrical section can have an elongated body section with two open ends. The elongated body section can be sized and shaped to slide over an ampule and the first open end can be sized and shaped to leave the discharge end of the ampule exposed. Other characteristics of the layer can be the same as that of the embodiment of FIG. 24. Once the layer is heated and wrapped tightly or snuggly around an ampule, the discharge end sleeve may be placed over the end of the ampule. Thus, when the outer layer is mounted over an ampule, the combination of the present embodiment can have at least three different materials—one being from the ampule itself, the second from the shrink wrap material, and the third being from the discharge end sleeve.

In another embodiment, a roll of shrink wrap sheet or layer can be used as an outer layer. The layer from the roll may be cut down to a working size sheet, applied over an ampule, and then subjected to heat to set over the ampule. The working size sheet may be rectangular in shape having a length and a width. Alternatively, the roll may be made as a stretch wrap layer or material. Stretch wrap materials are highly stretchable plastic films. The elastic recovery of each film keeps the item that the film is applied against tightly bound. The layer may be cut down to size, stretched and applied over an ampule to keep a tight fit over the ampule.

A needleless injector device or assembly of the present disclosure can be understood to include an ampule body, a plunger with a plunger tip, a piston with a piston head and a piston stem, a spring or motive force for propelling the piston head into the plunger, a safety lock, and an end cap for closing off the proximal opening of the elongated housing body. The cap can be removable following use to enable removable of the internal components, as previously discussed. In other examples, the cap can be integrated or unitarily formed with the elongated body. In still other examples, other components can be included or omitted.

In an example, the trigger can have a push end that is pointed in the proximal direction, towards the end cap or towards the proximal end of the injector body. In other embodiments, the push end of the trigger points distally towards the ampule. This may be arranged by changing the location of the cradle for holding the pivot pin that allows the trigger to rotate to release the spring. The present arrangement of the push end is ideally suited for use in applications that require the user to reach into a closed or confined space. For these applications, the user can still have access to the push end of the trigger to perform an injection. For example, when used in a dental application, such as to perform a local anesthesia or lidocaine injection, the direction of the push end allows the clinician to insert the ampule into the mouth to inject the gum near the molars while still allowing for easy reach or access to the trigger to release the spring and perform the injection.

In practice, the size of the injector assembly may be modified for different applications. For example, for dental applications or for small dosage applications, the size of the discharge end and injector end may be reduced. Furthermore, while the assembly is described for use with a confined space, it is not so limited and may be used in any wide open space application where subcutaneous delivery of medication is desired.

Methods of making or forming components of the injector assemblies and cover sheets discussed herein as wells as completed injector assemblies discussed herein are understood to be within the spirit and scope of the present disclosure. Methods of using components of the injector assemblies and cover sheets discussed herein as wells as using the completed injector assemblies discussed herein are also understood to be within the spirit and scope of the present disclosure. Still furthermore, the present needleless injector assemblies are also ideally suited for being pre-filled and packaged inside a blister pack or package so that a user simply has to pry open the package, placed the discharge end of the nozzle against the skin, and squeeze the trigger to deliver a dosage of fluid subcutaneously. In another example, only the ampule end is pre-filled and packaged inside a blister pack.

Aspects of the present disclosure include an ampule comprising: a body, a proximal opening at one end of the body, and a discharge end comprising a discharge tip having a nozzle in communication with an interior wall surface defining an interior cavity. A plunger is disposed at least partially within the interior cavity of the body, the plunger comprising a plunger tip in dynamic sealing arrangement with the interior wall surface of the body. A quick connect engagement mechanism is provided on the body. The quick connect comprising at least two spaced apart tapered engagement surfaces formed exteriorly of body; and wherein the body is made from a cyclo-olefin-copolymer material.

A further aspect of the present disclosure includes a drive end for a needleless injector device. The drive end comprising: an elongated body comprising a distal end comprising a distal opening, a proximal end comprising a proximal opening, an interior surface defining an interior cavity, and a channel disposed exteriorly adjacent the proximal opening, a trigger pivotably disposed about an exterior of the elongated body comprising a latch pin holding a piston against an expansion force of a spring, said piston comprising a shoulder in contact with the spring; an end cap engaging the channel of the elongated body to close the proximal opening; and wherein the spring exerts a spring force against an interior face of the end cap.

The injector device can further comprise an ampule attached to the distal opening of the elongated body.

The injector device can further comprise a plurality of spaced apart tines at the distal opening of the elongated body for connecting with an ampule.

The end cap can comprise a flange for engaging the channel and a second flange for engaging a second channel disposed exteriorly adjacent the proximal opening of the elongated body.

The channel and the second channel can be tapered so that the flange and the second flange are loosely held therein and are pivotable therein.

The injector device can further comprise a boss and a pin for securing the cap to the elongated body.

The pin can be located on the elongated body.

The injector device can further comprise an outer layer disposed around the ampule.

The outer layer can comprise an end wall comprising an opening and wherein the opening on the end wall is disposed around a nozzle on the ampule.

A still further aspect of the present disclosure is a needleless injector device. The needleless injector device comprising an ampule and a drive end. The ampule comprising a body made from a cyclo-olefin-copolymer material, a proximal opening at one end of the body, and a discharge end comprising a discharge tip having a nozzle in communication with an interior wall surface defining an interior cavity; a plunger disposed at least partially within the interior cavity of the body, the plunger comprising a plunger tip in dynamic sealing arrangement with the interior wall surface of the body of the ampule. The drive end comprising an elongated body comprising a distal end comprising a distal opening having the plunger projecting therethrough, a proximal end comprising a proximal opening, an interior surface defining an interior cavity, and a channel disposed exteriorly adjacent the proximal opening, a trigger pivotably disposed about an exterior of the elongated body comprising a latch pin holding a piston against an expansion force of a spring, said piston comprising a shoulder in contact with the spring; an end cap engaging the channel of the elongated body to close the proximal opening; and wherein the spring exerts a spring force against an interior face of the end cap.

Aspects of the present disclosure include a needleless injector assembly comprising: an ampule and a drive end. The ampule comprises a body, a proximal opening at one end of the body, and a discharge end comprising a discharge tip having a nozzle in communication with an interior wall surface defining an interior cavity. A plunger is disposed at least partially within the interior cavity of the body of the ampule. The plunger comprising a plunger tip in dynamic sealing arrangement with the interior wall surface of the body. A quick connect engagement mechanism is located at a proximal end of the body comprising a flange engaged to a connection mechanism on the drive end, which comprises a spring. The connection mechanism can comprise a plurality of spaced apart tines. Wherein the drive end comprises an end cap removably engaged to an exterior surface a housing body of the drive end, the spring applying a first force against the end cap when cocked and applying a second lower force against the end cap when no longer cocked.

The needleless injector assembly can further comprise a sleeve mounted over the discharge end of the ampule.

The sleeve can extend proximally over the body of the ampule.

The sleeve can further comprise a body sleeve mounted over the body of the ampule and spaced from the sleeve.

A further aspect of the assembly comprises closing ring for closing the tines over the quick connect engagement mechanism on the body.

A further aspect of the present device, system, and method include a drive end for a needleless injector device comprising an elongated body comprising a distal end comprising a distal opening and a plurality of spaced apart tines, a proximal end comprising a proximal opening, an interior surface defining an interior cavity, and a channel disposed exteriorly adjacent the proximal opening. A trigger can be mounted pivotably to the elongated body and disposed about an exterior of the elongated body comprising a latch pin or trigger finger holding a piston against an expansion force of a spring. The piston can comprise a shoulder in contact with the spring and an end cap can engage the channel of the elongated body to close the proximal opening; and wherein the spring exerts a spring force against an interior face of the end cap.

The drive end can further comprise an ampule comprising a flange mechanically coupled to the plurality of tines.

A still further aspect of the present disclosure is a method for separating metallic from thermoplastic components in a needleless injector device or assembly comprising the steps of sliding an end cap radially relative to a longitudinal length of a drive end; exposing a proximal opening of the drive end; and removing the spring from the drive end.

Yet another aspect of the present disclosure is a method for manufacturing a needleless injector assembly. As disclosed, the method can comprise the steps of forming an ampule from a first material, the ampule comprising an elongated body having an exterior surface, an interior surface, an open proximal end, and a distal wall comprising a nozzle having a lumen passing through the distal wall and placing a plunger comprising a plunger tip in dynamic sealing arrangement with the interior surface of the ampule. The method can further include the steps of forming an injector body comprising an elongated injector body comprising a distal end comprising a distal opening having the ampule attached thereto, a proximal end comprising a proximal opening, an interior surface defining a bore with an interior cavity, and a channel disposed exteriorly adjacent the proximal opening; placing a piston and a spring inside the bore of the elongated injector body; mounting a trigger to the elongated injector body, the trigger comprising a latch pin for holding the piston against an expansion force of the spring; and placing an end cap at the proximal end of the elongated injector body to close the proximal opening; wherein the end cap is pivotable about a lengthwise axis of the injector body when not under full biasing force of the spring. In some examples, an outer layer having an elongated body made of a second material can be disposed over the elongated body of the ampule and wherein the outer layer comprises a length and an inside diameter.

The method can further comprise pushing the piston to compress the spring prior to attaching the ampule to the elongated injector body.

The method can further comprise heating the elongated body of the outer layer to shrink the length or the inside diameter.

The method wherein the cap can have two inside flanges for engaging two corresponding channels on the elongated injector body.

A still yet further feature of the present disclosure include an impact resistant ampule for use in a needleless injection. The impact resistant ampule comprises a body comprising an interior surface and an exterior surface, a proximal end comprising a proximal opening at one end of the body, and a discharge end comprising a discharge tip having a nozzle in communication with the interior surface of the body, which defines an interior cavity; wherein the body is made from a first material and a plunger disposed at least partially within the interior cavity of the body, the plunger comprising a plunger tip in dynamic sealing arrangement with the interior wall surface of the body. The ampule can include a connect engagement mechanism at the proximal end for connecting to a spring injector. An outer layer comprising an elongated body having a length and a bore made from a second material is disposed over and tightly fitting around the exterior surface of the body so as not to slip off of the body. Preferably, the first material is more rigid than the second material.

The connect engagement mechanism can comprise at least two spaced apart tapered engagement surfaces formed exteriorly of the body.

The outer layer can be made from a shrink wrap material having an inside diameter that is larger than an outside diameter of the body prior to being subject to heat.

The outer layer can include an elongated body comprising an open proximal end and an open distal end.

The outer layer can have an elongated body comprising an open proximal end and a distal wall comprising a distal opening having a perimeter that is smaller in dimension than an inside diameter of the elongated body.

The impact resistant ampule can further comprise an injector end connected to the ampule, the injector end comprising an injector body defining a bore having a piston pushing against a spring located therein to compress the spring and wherein a latch pin operatively mounted to the injector body abuts the piston to hold the spring in a compressed state.

The impact resistant ampule can further comprise a trigger having a push end for triggering to release the spring; wherein the push end of the trigger faces a proximal end of the injector body and away from a distal end of the injector body to facilitate triggering by a user.

A blister pack can be provided comprising a cavity having a peelable cover placed around the ampule. In other words, the ampule can be packaged inside a blister pack.

Method of making and of using a combination needleless injector device and cover sheet are within the scope of the present invention.

A cover sheet as shown and described herein.

A still yet further aspect of the present disclosure includes a method of delivering a therapeutic amount of fluid subcutaneously without a needle. The method can comprise depressing a trigger on a needleless injector device comprising an ampule an injection driver at an injection site, said ampule comprising a nozzle at a discharge end and a slidable plunger and said injection driver comprising a spring and a piston; applying a cover sheet over the injection site, said cover sheet comprising an impermeable layer comprising an outer surface side and an inner surface side that faces the injection site, said cover sheet comprising a continuous adhesive ring located on the inner surface side of the impermeable layer; and wherein said adhesive ring defines a region external of the adhesive ring without adhesive and a region internal of the adhesive ring without adhesive.

The method can further comprise prepping the injection site by wiping with an alcohol wipe or an antiseptic wipe prior to depressing the trigger.

The cover sheet can be removed from a water impermeable overwrap prior to applying the cover sheet over the injection site.

The cover sheet can be removed from a bulk package comprising a plurality of individually wrapped cover sheets.

T cover sheet can have a shape that is round, oval, square, or rectangular.

The adhesive ring on the cover sheet can have a shape that is round, oval, square, or rectangular.

The cover sheet can comprise an inner layer adhered to the impermeable layer and the adhesive ring adhered to the inner layer.

The therapeutic amount for delivering subcutaneously can be hyaluronic acid.

The inner layer of the cover sheet can comprise an alginate dressing.

A still yet further aspect of the present disclosure is a combination needleless injector device and cover sheet comprising: an ampule comprising a body having a discharge end with at least one nozzle and a plunger slidably disposed inside the body; an injection driver comprising an elongated body comprising a distal end comprising a distal opening having the ampule attached thereto and an interior surface defining an interior cavity having a spring and a piston located therein; a trigger pivotably disposed about an exterior of the elongated body comprising a latch pin holding the piston against an expansion force of the spring, said piston comprising a shoulder in contact with the spring; a cover sheet wrapped inside a liquid impermeable overwrap, said cover sheet comprising an impermeable layer comprising an outer surface side, an inner surface side, and a continuous adhesive ring located on the inner surface side of the impermeable layer; and wherein said adhesive ring defines a region external of the adhesive ring without adhesive and a region internal of the adhesive ring without adhesive.

The combination can further comprise an outer layer disposed around the ampule.

The outer layer can comprise an end wall comprising an opening and wherein the opening on the end wall can be disposed around the at least one nozzle on the ampule.

A cover sheet for use with a needleless injector device comprising: a liquid impermeable layer with adhesive located inside a liquid impermeable overwrap, said liquid impermeable layer comprising an outer surface side, an inner surface side, and a continuous adhesive ring located on the inner surface side of the impermeable layer; and wherein said adhesive ring defines a region external of the adhesive ring without adhesive and a region internal of the adhesive ring without adhesive.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is a schematic side view of a needleless injector device provided in accordance with aspects of the present disclosure.

FIG. 2 is a perspective exploded view of the needleless injector device of FIG. 1, which shows the discharge end separated from the drive end.

FIG. 3 is a schematic partial cross-sectional side view of the needleless injector device of FIG. 1, which shows a motive force in a ready to use state to propel a piston.

FIG. 4 is a schematic partial cross-sectional side view of the needleless injector device of FIG. 3 with the safety lock released so that the trigger may be pressed to deliver a fluid.

FIG. 5 is a schematic partial cross-sectional side view of the needleless injector device of FIG. 4 with the trigger depressed to release the spring and the spring released to propel the piston.

FIG. 6 is an expanded view of the proximal end of the drive end, which shows the end cap engaged to the elongated body and a secure pin projecting into a secure boss when the spring is compressed.

FIG. 7 is an expanded view of the proximal end of the drive end, similar to FIG. 6, which shows the end cap engaged to the elongated body and a secure pin projecting into a secure boss when the spring is released.

FIG. 8 is a schematic partial cross-sectional side view of the needleless injector device of FIG. 5 with the end cap partially removed from the proximal end of the elongated body of the drive end.

FIG. 9 is a schematic partial cross-sectional side view of the needleless injector device of FIG. 5 with the end cap completely removed from the proximal end of the elongated body of the drive end.

FIG. 10 is a cross-sectional side view of a cover sheet in accordance with aspects of the present disclosure.

FIG. 11 is a cross-sectional side view of an alternative cover sheet in accordance with aspects of the present disclosure.

FIG. 12 is a bottom view of the cover sheet of FIG. 10 with the release layer removed therefrom.

FIG. 13 is a process flow diagram depicting a method of using a combination needleless injector and cover sheet of the present disclosure.

FIG. 14 is a perspective exploded view of an alternative needleless injector device, which shows a discharge end separated from a drive end.

FIG. 15 is an exploded perspective view of a drive end of a needleless injector assembly provided in accordance with further aspects of the present disclosure.

FIG. 15A is a perspective view of a piston provided in accordance with the present disclosure.

FIG. 16 is an assembled view of the drive end of FIG. 15.

FIG. 17 is a further assembled view of the drive end of FIG. 15.

FIG. 18 is a completed assembled view of the drive end of FIG. 15.

FIG. 19 is a perspective view of an ampule with plunger in the process of being mounted to the drive end of FIG. 18.

FIG. 20 is an assembled perspective view of a needleless injector assembly provided in accordance with aspects of the present disclosure.

FIG. 21 is a perspective view of an ampule and plunger with different sleeve embodiments.

FIG. 22 is a perspective view of the ampule of FIG. 21 with a discharge end sleeve and body sleeve positioned over the body of the ampule.

FIG. 23 is a perspective view of the ampule of FIG. 21 with one-piece sleeve positioned over the body of the ampule.

FIG. 24 is a perspective view of an alternative outer layer for placing over an ampule.

FIG. 25 is a perspective view of another alternative outer layer for placing over an ampule.

FIG. 26 is a perspective view of a roll of shrink wrap sheet or stretch wrap sheet for use with an ampule.

FIG. 27 is a cross-sectional side view of a needleless injector assembly provided in accordance with further aspects of the present disclosure in a ready to use position.

FIG. 28 is a cross-sectional side view of the needleless injector assembly of FIG. 27 with the spring being released or following released of the spring.

### DETAILED DESCRIPTION

The detailed description set forth below in connection with the appended drawings is intended as a description of the presently preferred embodiments of needleless injectors provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

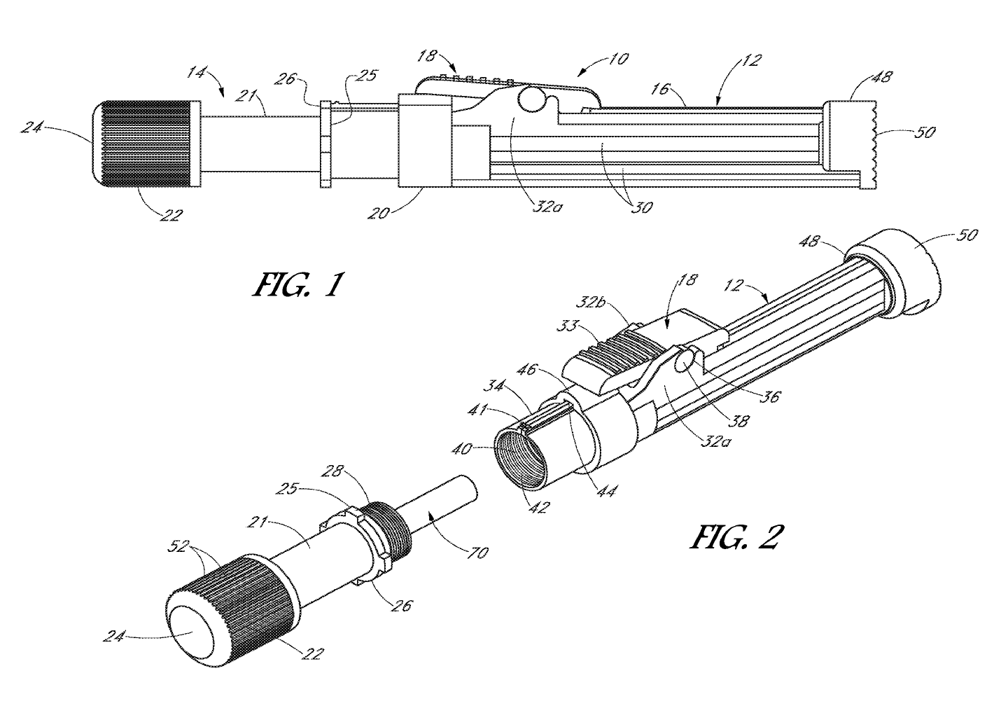

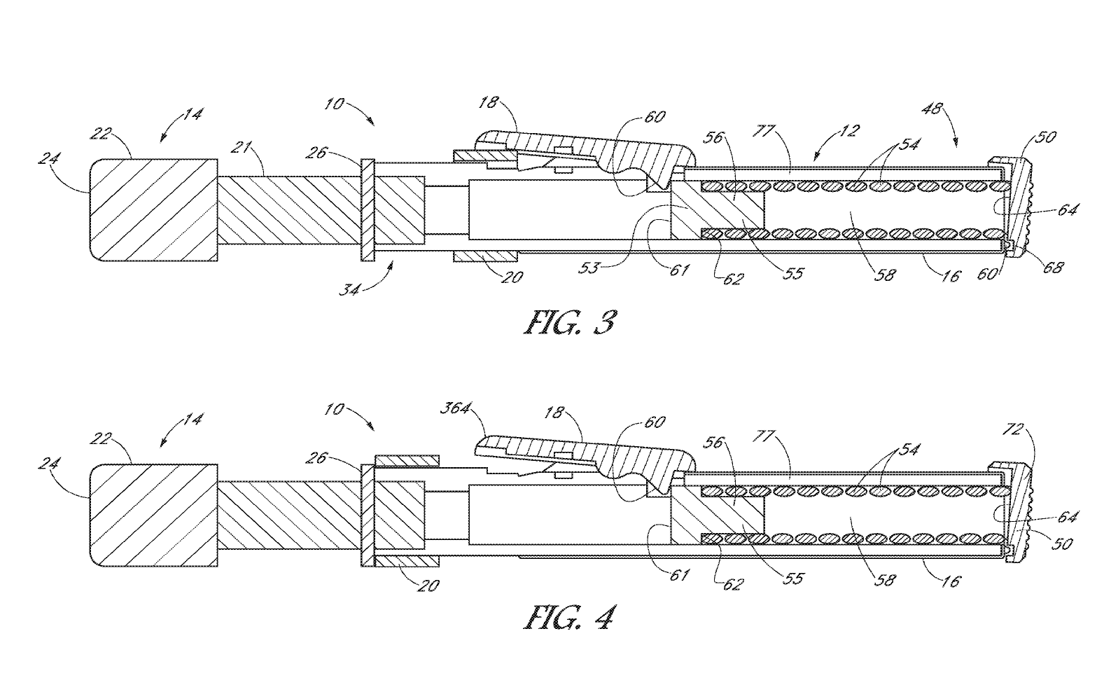

FIG. 1 is a schematic side view of a needleless injector device or needleless injector assembly provided in accordance with aspects of the present system, device and method, which is generally designated 10. Broadly speaking, the needleless injector device 10 comprises a drive end component 12, or drive end for short, and a discharge end component 14, or discharge end for short. The drive end 12, also sometimes refers to as an injection driver, injector end, spring end or simply injector, comprises an elongated body or housing 16 comprising a trigger 18 for holding back and subsequently releasing a motive force located inside the elongated body 16 when discharged to propel a piston (See 56 of FIGS. 3 and 4), also located inside the elongated body, to then propel a plunger (70 of FIG. 2) located inside the discharge end 14, as further discussed below. In one example, the drive end 12 comprises a safety mechanism or safety lock 20 that is displaceable to allow the trigger 18 to be activated. As shown, the safety lock 20 is a ring that is slidable about the elongated body 16 to unlock the trigger, such as to provide space for the trigger to be depressed. In other embodiments, the safety lock 20 is rotatable or pivotable to unlock the trigger, such as to provide space for the trigger to move, pivot or rotate. In still yet other examples, the safety lock 20 is both slidable and rotatable to unlock the trigger. In other examples, the safety feature comprises a frangible tab that is removable to provide space for the trigger 18 to be depressed or released.

The discharge end 14 is configured to hold a volume of fluid, such as a fluid medicament, vaccine, flu shot, insulin, local anesthesia, lidocaine, hyaluronic acid, tetanus shot, etc., for subcutaneous delivery to a patient. For example, the discharge end can contain a clinically effective amount of hyaluronic acid for injecting the facial area for cosmetic treatment. As shown, the discharge end 14 is an ampule comprising a discharge head 22 having a discharge tip 24 and a discharge base 25 having a flange 26 and a coupling end 28 (FIG. 2), which in the present embodiment comprises a threaded end. The discharge head 22 is shown relatively larger in cross-sectional dimension than the body 21. In other examples, the two are the same or the body is larger. In other examples, the coupling end 28 is a quick release end without threads. The discharge end 14 is configured to threadedly engage with or to the drive end 12. In other examples, the ampule has a quick release cam surface for engaging a mating surface on the discharge end, as further discussed below. The discharge end 14, for example an ampule, has an elongated body 21 of a size and dimension for sufficiently holding a desired volume of fluid medicament for subcutaneous delivery to a patient. In some examples, the assembly 10 is sized for use in dental applications, such as for delivering local anesthesia to the gum or mouth.

In one example, the discharge end 14 is made from a cyclo-olefin-copolymer (COC) material, such as from TOPAS and APEL Mitsui Chemical of Japan. It is believed that fluid medicament may be stored in the ampule made from COC for a much longer period than for ampules made from other thermoplastic or engineered plastic materials. This allows for the ampules to be pre-filled and stored with different fluid medicaments and refrigerated so that they may be readily available for use with drive ends of the present disclosure. In still other examples, the discharge end 14 is made from a plastic material, such as a thermoplastic material, selected to have impact resistant characteristics. For example, the discharge end may be made from plastic injection molding using an acrylic-based polymer, such as ACRYLITE® and HYGARD®, the latter being made from a multi-layer of polycarbonate and acrylic. Optionally, the discharge end 14 may be made from plastic injection molding using a polycarbonate (PC)-based material, such as LEXAN®, MARKOLON®, SAFEGUARD®, and SAFEGUARD HARDCOAT®. In still other examples, the discharge end 14 may be made from plastic injection molding using a polyethylene (PE)-based material, such as POLYSTONE® PG100, POLYSTONE® 500, and POLYSTONE® MATROX.

With reference again to FIG. 1, the drive end 12 is preferably made from a hard plastic, such as high density polyethylene (HDPE), polycarbonate (PC), polyvinyl chloride (PVC), COC or other comparable hard plastic. In one example, the elongated body 16 is made from two separate housing halves, such as by plastic injection molding two different opaque sections, that are joined together along a lengthwise seam by welding, gluing, detents, or combinations thereof. The elongated body 16 may comprise a plurality of ribs 30, such as elongated ribs, that extend at least partially along the length of the elongated body. In other examples, the body has a smooth outer surface contour, a plurality of bumps or projections, or combinations thereof. A pair of mounting flanges 32a, 32b are provided near the distal end 34 of the drive end 12 with each comprising a cradle 36 for receiving or accommodating a pivot pin or shaft 38. The opening of each cradle 35 can be sized and shaped to receive the pivot pin or shaft 38 in a snap fit arrangement.

The pivot shaft 38 is operatively connected to the trigger 18 so that when the trigger is pushed, it rotates about the pivot shaft 38. In some examples, the trigger 18 and the pivot shaft 38 are unitarily formed, such as by injection molding, co-molding, or insert molding. The trigger 18 is shown with a plurality of exterior gripping features 33, which are ribs, projections, or bumps formed on the outer surface to facilitate gripping. In other examples, a pin is connected or mounted with the body 16 of the discharge end 12 and the trigger 18 is equipped with a pair of cradles (similar to cradle 36) for snapping onto the pin mounted to the injector body 16.

FIG. 2 is an exploded perspective view of the assembly 10 of FIG. 1. As shown, the drive end 12 comprises a distal opening 40 comprising a threaded bore 42 for receiving the threaded end 28 of the discharge end 14, such as the ampule. Also shown is a rail or track 41 at the distal end 34 of the drive end 12 for accommodating a channel 44 formed in the interior bore of the safety lock 20 to ride there-along and for rotational alignment. The track 41 ensures the ring is rotationally aligned so that a protrusion or raised bump 46 formed on the exterior surface of the safety ring 20 aligns with the trigger 18 so as to provide a physical presence under the trigger to prevent the trigger from triggering until the obstruction is removed. In other examples, the raised bump 46 formed on the safety ring 20 is rotatable from a position away from the trigger 18 to a position below or under the trigger 18 to provide the physical barrier for preventing triggering. In yet other examples, the trigger 18 is provided with a collapsible or frangible leg that severs or collapses upon exertion of a sufficient downward pressure on the trigger.

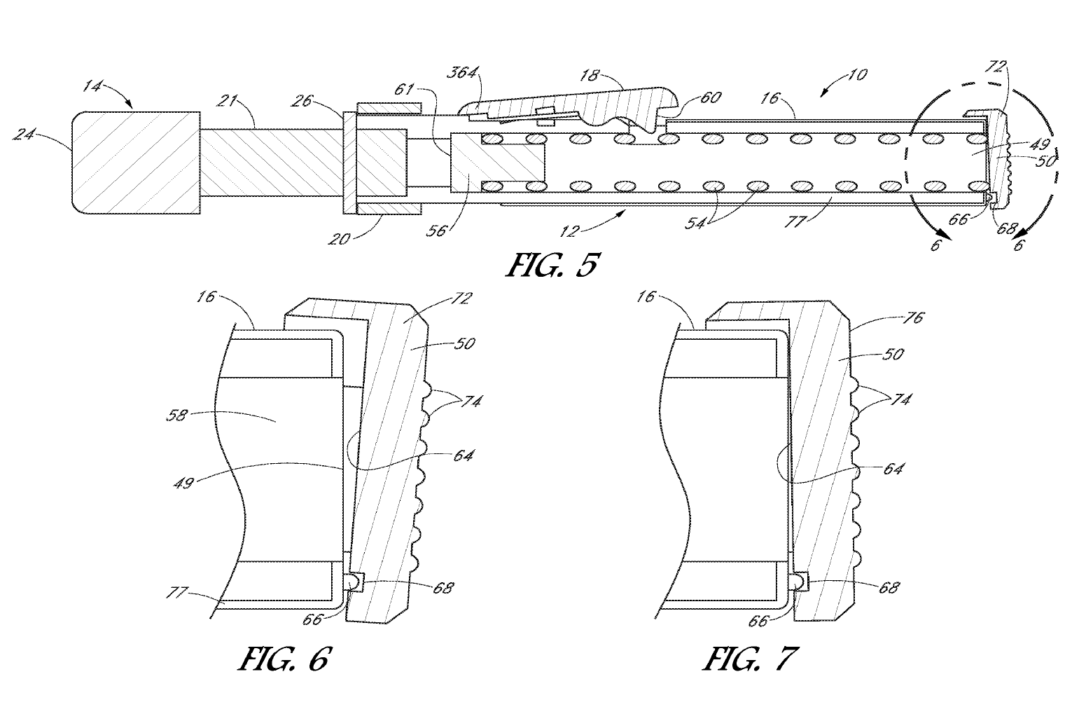

An end cap 50 is provided at the proximal end 48 of the drive end 12. The end cap 50 is provided to cover or close-off the proximal opening 49 (FIG. 5) of the elongated body 16 after installation of various injector components. In one example, the end cap 50 is threadedly engaged to threads located on the elongated body 16. In another embodiment, as further discussed below, the end cap 50 is provided with a slidable mechanism for engaging corresponding features located at the proximal end 48 of the elongated body 16 to close the proximal opening 49 of the elongated body. For example, the end cap 50 can comprise an end wall and a rim having an open passage through the rim so that the cap can slide over the proximal opening via the passage through the rim. Flanges can be formed on the rim of the cap to then engage tracks or channels on the elongated body 16 to secure that the cap is attached thereto and prevent from displacing in the axial by the force of the spring. Said differently, physical restraints can be employed between the elongated body and the cap, such as sliding rails, tongue-groove, etc., to ensure engagement. A detent, such as a pin and a boss, may be used to then secure the cap to the elongated body from being displaced radially relative to the lengthwise axis of the body 16 after the initial engagement. The slidable mechanism between the cap 50 and the elongated body 16 permits easy subsequent removal of the end cap from the elongated body to expose the proximal opening 49 to facilitate optional removal of the spring located inside the elongated body 16.

As shown in FIGS. 1 and 2, the ampule 14 includes an enlarged discharge head 22, which is larger in outside diameter than the outside diameter of the elongated body 21. The enlarged discharge head 22 has a generally constant outside diameter along a length of about 10% to about 35% of the total length of the ampule 14. Optionally, the wall thickness of the elongated body 21 is constant and the enlarged discharge head is omitted. Exteriorly, the discharge head 22 comprises a plurality of generally parallel fins 52 to facilitate gripping when mounting the discharge end 14 onto the drive end 12.

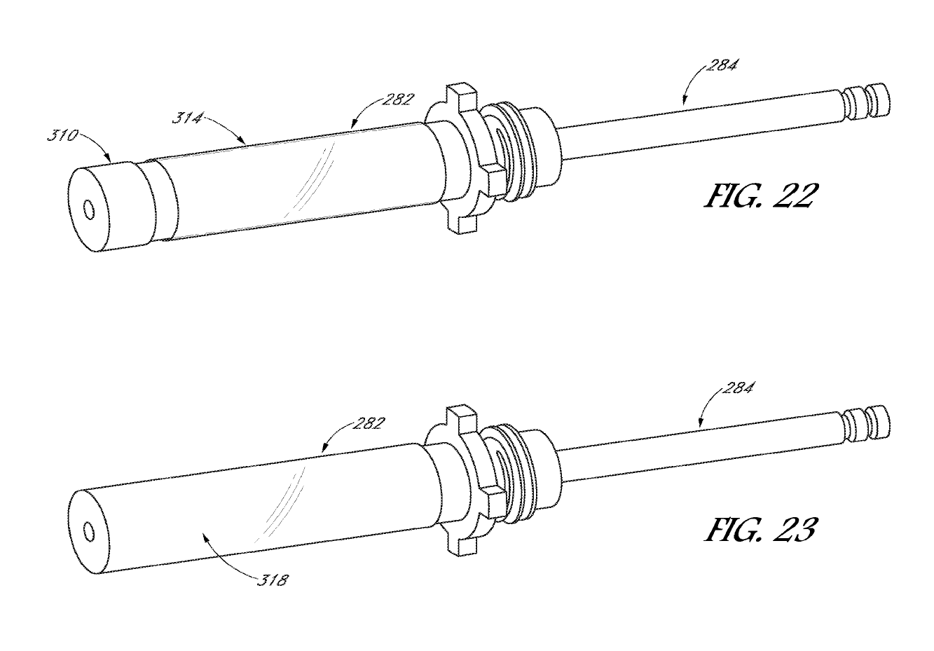

Although not shown, at least one outlet nozzle is provided at the discharge tip 24. The outlet nozzle, which can have a diameter or bore size in the range of four thousandths to twelve thousandths, is sized and shaped to allow fluid inside the ampule 14 to be discharged therethough to subcutaneously deliver an injection of fluid to a patient. To facilitate discharging fluid out the nozzle of the ampule 14, a plunger 70 is slidably provided in the interior cavity of the ampule (See also FIGS. 23 and 24). When the plunger 70 is advanced, such as pushed by a spring following activation of the trigger 18, the plunger tip pushes fluids inside the ampule out the discharge nozzle at the discharge tip 24. The plunger 70 has a plunger tip (FIGS. 23 and 24, 285) for dynamically sealing against the interior surface of the ampule to discharge fluid out the nozzle, similar to a plunger inside a barrel of a syringe. Further aspects of prior art needleless injectors are disclosed in U.S. Pat. Nos. 6,558,348; 5,704,911; 5,569,189; and 5,499,972. To the extent structures and features disclosed in these four prior art patents do not conflict with expressly disclosed features of the present disclosure, they are expressly incorporated herein by reference for their teachings.

FIG. 3 shows a schematic partial cross-sectional side view of the needleless injector assembly 10 of FIGS. 1 and 2 with a helical spring 54 and a piston 56 positioned inside the interior cavity or bore 58 of the elongated body 16 of the drive end 12. The piston 54 is shown with a piston head 53 and a piston stem 55 defining a shoulder 62 therebetween. Both the piston head and the piston stem can be annular or round in nature, along an end cross-section. The length of the stem 55 and the length or thickness of the piston head 53 can vary. The spring 54, which is made from a metal, such as from carbon steel, is shown in a compressed state with the piston 56 moved proximally of the latch pin 60 located on the trigger 18, which abuts the piston face 61 to hold the piston 56 which then holds the spring 54 in compression. The spring 54 may be compressed or set to compress in the manner shown and described in the '911, '189 and '972 patents, such as by using a setting tool to push against the piston head 53 to compress the spring and allowing the latch pin 60 to move distal of the piston face 61 to retain the spring in the compressed or loaded position. In the example shown, the latch pin 60 is a projection formed integrally or unitarily with the trigger 18. In another example, the latch pin is separately formed and subsequently attached to the trigger 18. The latch pin 60 can have a generally flat or planar surface on the proximal side for abutting the piston face 61.

The compressed spring 54 exerts a high spring force against both the shoulder 62 on the piston 56 and the interior surface 64 of the end cap 50. As the end cap 50 can pivot due to a gap or slack in the mounting channel with the elongated body 16, as further discussed below, the spring force causes a secure pin 66 located at the proximal end 48 of the body 16 and a secure boss 68, similar to a bore or a recess, located on the end cap 50 to engage. In other words, the spring forces the cap to pivot relative to the proximal end of the elongated body, which then causes the secure pin 66 to engage the secure boss 68 to ensure retention of the cap to the body. This engagement prevents the cap from sliding radially to separate from the engagement between the flanges on the cap and the channels on the elongated body 16. This feature allows the end cap 50 to latch onto the elongated body 16 and held at the proximal end 48 of the elongated body without threads. In other examples, the cap has two channels and the elongated body 16 has two flanges that engage the channels to secure the cap to the body 16. The spring force acting on the end cap 50 provides added resistance against potential unlatching between the secure pin 66 and the boss 68. For example, the spring force causes the pin 66 to engage the secure boss 68 to prevent sliding the cap radially relative to the lengthwise axis of the body until the spring force is reduced or removed. In other examples, the cap 50 has a pin and the elongated body 16 has a secure boss 68. In still other examples, the cap 50 is threaded to the body 16 and held there by the threaded engagement with further securement provided by the force of the spring acting on the interior surface 64 of the cap. Note that FIG. 3, as well as other figures shown herein, does not show a plunger 70 (FIG. 2) located inside the ampule for clarity. In practice, a plunger 70 is slidably positioned inside the ampule and the piston 56 is configured to push a proximal end of the plunger 70 when the trigger 18 is depressed to release the spring 54, which then forces the piston 56 against the proximal end of the plunger to propel the plunger 70 in the distal direction to expel fluid inside the ampule out the one or more nozzles at the discharge end 24 of the ampule.

FIG. 4 depicts the assembly of FIG. 3 with the safety lock 20 moved forward or distally to move the exterior protrusion 46 (FIG. 2) on the safety lock 20 away from the trigger 18 and provide clearance between the trigger and the exterior surface of the body 16 for triggering.

FIG. 5 depicts the assembly of FIG. 4 with the trigger 18 depressed at the push end 364 to release or move the latch pin 60 away from the piston face 61, such as to move the latch pin 60 radially away from the lengthwise axis of the elongate body 16. As shown, the push end 364 of the trigger 18 is arranged to face or point in the distal direction, i.e., points towards the ampule. This pushing of the trigger 18 at the push end releases the spring 54 and allows the spring to rapidly expand to propel the piston 56 into the plunger 70 (FIG. 2) to then propel the plunger into the interior of the discharge end component 14 to expel fluid out the nozzle at the discharge tip 24 of the discharge end or ampule 14. Consequently, an injection can be made by placing the discharge tip 24 of the injection assembly 10 against the skin of a patient, pulling or depressing the trigger 18 to release the spring 54 to then push the piston 56 against the plunger 70 (FIG. 2) and discharging fluid medicament held inside the hollow cylinder 21 of the discharge end 14 into the patient.

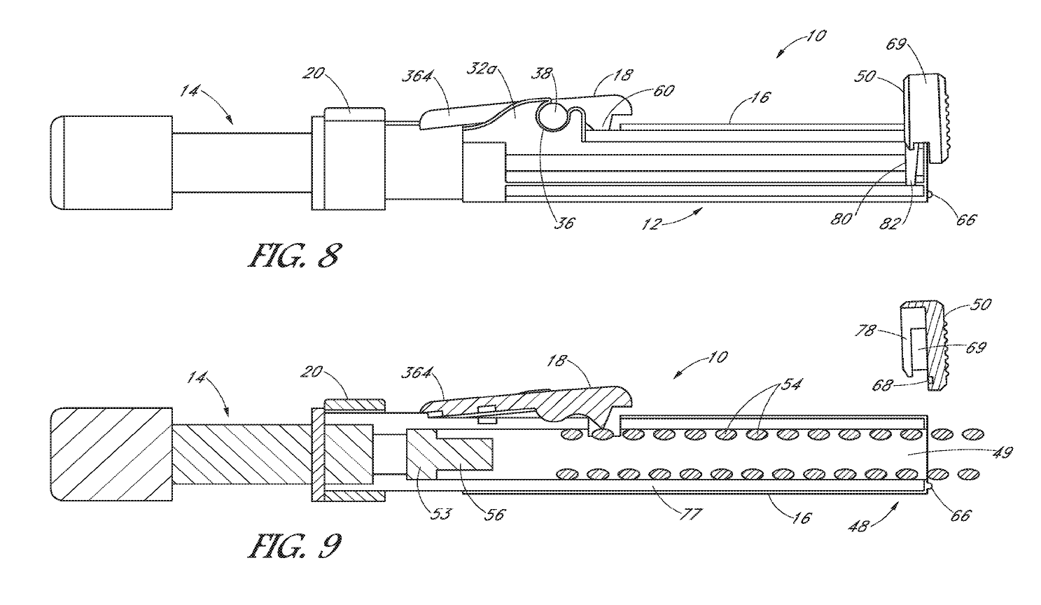

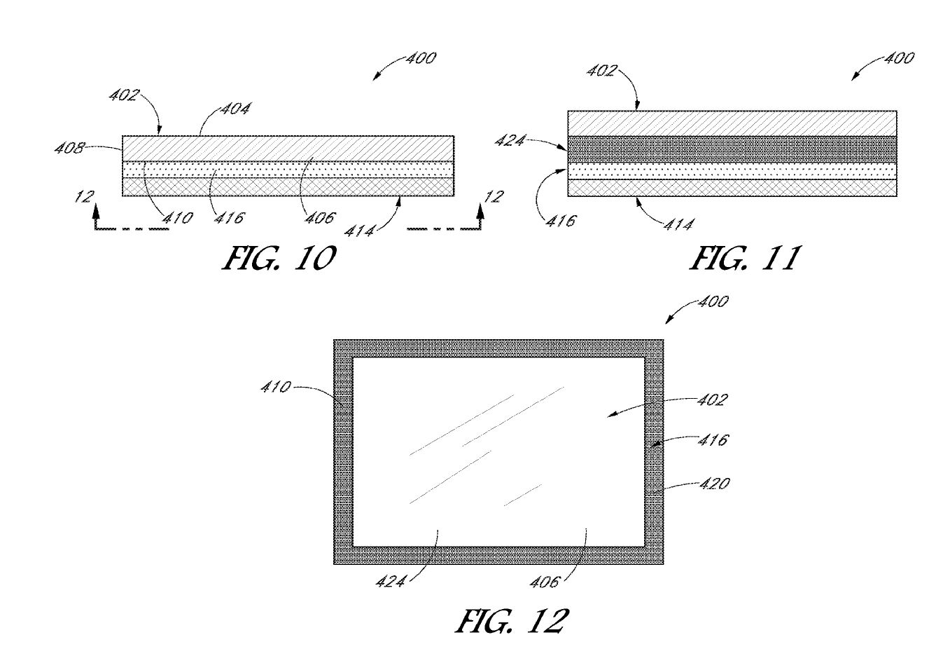

FIGS. 6 and 7 are expanded views of FIG. 5 taken at exploded point “A” shown without the spring 54 and shown under two different situations, when biased by the spring to cause engagement (FIG. 6) and when the bias force is reduced or removed (FIG. 7). With reference to FIGS. 6 and 7 in addition to FIG. 5, the expanded spring 54, after fluid discharge, exerts a lower spring force on the interior surface 64 of the end cap 50 than when the spring is in the compressed state, as shown in FIGS. 1 and 3. Consequently, less force is exerted on the end cap 50 by the spring after the spring is released and the cap is less torqued or slanted about its upper end 72, which is shown in the expanded view of FIG. 7. This less slanted position may further be facilitated by pushing on the cap near element 72. The manner in which the cap 50 engages the body 16 using a slidable mechanism is further discussed below with reference to FIGS. 8 and 9. Thus, as shown in FIG. 7, the cap 16 is positioned generally square or vertical relative to the end edge of the body 16 when the spring is released.

In contrast, when the spring 54 is compressed as shown in FIGS. 3 and 4 and ready to be released by depressing the trigger 18, the spring force exerts a greater outward force on the end cap 50 and causes it to slant to force the secure pin 66 and the secure boss 68 to engage, which is shown in the expanded view of FIG. 6. The lower load on the end cap 50, after the spring expands as shown in FIG. 7, allows a user to manipulate the cap to separate it from the drive end 12. For example, when the secure pin 66 and the secure boss 68 are not engaged, as shown in FIG. 7, the end cap 50 can readily slide radially to separate the cap from the elongated body. Removal of the end cap 50 allows the spring 54 to be removed from the elongated body 16 through the proximal opening 49 of the elongated body. This allows a user to separate a metal component, i.e., the spring, from the various thermoplastic components. The components of the needleless injector 10, after the spring 54 is removed, are all or are mostly non-metallic and therefore can easily be placed into a recycling bin for recycling. The separated spring 54 may be re-used, if desired, otherwise discarded in an appropriate bin for disposal or recycling.

In one example, the end cap 50 may be pushed in the distal direction near the upper end 72 of the cap with a finger or a thumb to further facilitate separating the secure pin 66 from the secure boss 68. For example, using a finger and pushing the cap 50 near point 72 can reduce the slanting of the cap relative to the body to separate the secure pin 66 from the retaining boss 68. The ability of the cap to cant or slant is provided by an engagement between a flange inside a tapered channel. The tapered channel, which is wider at one end than at another end, provides room for a flange to move within the channel. Once the pin 66 and the secure boss 68 are clear or spaced from one another, the end cap 50 can slide radially relative to the longitudinal axis of the device 10 to separate from the housing 16, as further discussed below. In an alternative embodiment, the secure pin 66 may be located on the end cap 50 and the secure boss 68 may be located on the elongated body 16, i.e., reverse from as shown. As shown, a plurality of spaced apart projections or protrusions 74 may be incorporated at the rear surface 76 of the end cap 50 to provide added traction for the finger or thumb when sliding and removing the end cap 50.

FIG. 8 is a schematic side view of the device of FIG. 1 showing the end cap 50 in the state of partial removal from the elongated body 16 of the injector end 12. FIG. 9 is a schematic cross-sectional side view of FIG. 8 with the cap further removed from the elongated body 16. As shown in FIG. 9, the end cap 50 incorporates two engagement flanges 78 (one shown) for mating engagement with respective channels 80 formed at the proximal end 48 of the elongated body 16. The flanges 78 are formed on the rim 69 of the cap 50. In one example, the channels 80 are each tapered by having a relatively large width at an end or edge of the body 16 with the trigger 18 and tapers inwardly as it proceeds downwardly towards the other edge of the injector body 16. If the parting line 77 defines a vertical plane running lengthwise with the injector assembly 10, one channel 80 is located on an outer surface of the body 16 on each side of the vertical plane. The cap 50 has a corresponding flange 78 located on the inside or interior of the cap 50 for engaging the exteriorly located channels 80. The tapered channel 80 narrows as it extends towards the terminal end or closed end 82 of the channel. When the cap 50 is placed over the proximal end 48 of the elongated body 16, the two internal flanges 78 on the cap 50 engage the two outer channels 80 on the elongated body 16. This prevents the cap from being displaced axially in the proximal direction (i.e., to the right of the figure). When the two engagement flanges 78 are fully engaged to the two channels 80, the end cap 50 closes off the proximal opening 49 of the elongated body 16 and the secure boss 68 and secure pin 66 cooperate to prevent the end cap 50 from being displaced therefrom, in addition to the spring force acting on the end cap to bias the pin and the secure boss to engage. The tapered channels 80 provide a degree of freedom by allowing the engagement flanges 78 on the cap to move within the confines of the channels, as discussed above with reference to FIGS. 6 and 7. This is especially true after the spring 54 has released and a lower spring force is acting on the end cap 50, as shown in FIG. 5.

FIG. 9 shows the end cap 50 completely separated from the elongated body 16 and the spring 54 extending partially out of the proximal opening 49. At this point, a user can easily remove the spring 54 and safely discard it, such by pulling the spring out of the elongated body with a hand or rotating the device 10 and pointing the proximal end down so that the spring 54 drops out of the proximal opening 49 under its own weight. Thus, the cap 50 allows for an environmental friendly device that facilitates separating metallic components from non-metallic components for recycling.

Although not shown, the piston 56 is preferably incorporated with features to prevent it from coming out the proximal opening 49 when the spring is being removed. In one example, the piston 56 is incorporated with a notch (not shown) on the enlarged drum or head 53 to prevent it from moving proximally of a plate or flange near the proximal opening. Other means for preventing the piston 56 from being displaced out of the proximal opening 49 may be used without deviating from the spirit of the present disclosure.

From the present disclosure, the present devices, systems, and methods are understood to include a needleless injector comprising an ampule and a plunger located in an interior cavity thereof connected to an injector driver comprising an elongated body, a piston, a trigger, a compression spring, and an end cap; wherein the end cap is pivotable or cant-able from a more vertical position to a more slanted position relative to the lengthwise axis of the elongated body. For example, the cap may define a plane that is at about 65 to 85 degrees from perpendicular with the lengthwise axis of the elongated body when the spring is held compressed. The cap 50 can then slant less from about 80 degrees to a generally vertical position at about 90 degrees with the lengthwise axis of the elongated body 16 when the spring 54 is no longer held compressed by any part of the trigger 18, whether directly or indirectly, such as when the spring expands following an injection. In a particular embodiment, a secure pin 66 and a secure boss 68 engage one another at the proximal end 48 of the elongated body 16 to secure the end cap 50 to the elongated body 50. The end cap 50 being removable from the elongated body 16 by separating the secure pin 66 from the secure boss 68. In one example, after the secure pin is separated from the secure boss, the end cap can slide radially relative to longitudinal length or axis of the elongated body 16 to separate the end cap 50 from the elongated body. This allows the user to readily remove various injector components from the elongated body to then recycle, re-use, and/or dispose.

The present disclosure is further understood to include an all elastic and/or thermoplastic needleless injector, other than for the spring, which can be made from a metal. Thus, upon removal of the spring from the elongated body, the needleless injector may be placed inside a recycling bin for non-metallic materials to be recycled. Features of the present disclosure are therefore understood to include an environmentally friendly device that is readily capable of recycling by allowing easy access to the components of the assembly to separate metallic from plastic components. In an example, this is facilitate by incorporating a cap with easy installation and removable to facilitate separation of the cap from the injector body for removal of metallic components from non-metallic components.

With reference now to FIG. 10, a cross-section side view of a cover sheet 400 in accordance with aspects of the present disclosure is shown. The cover sheet 400 may be made from a single ply or a multi-laminate layer. As shown, the cover sheet 400 comprises a fluid or liquid impermeable main layer 402 used as backing comprising an upper or outer surface 404 and a lower or inner surface 406 that faces the skin when the cover sheet 400 is applied onto the skin, as further discussed below. The main layer 402 has a perimeter 408 and an inner surface edge 410 adjacent the perimeter 408 for use with an adhesive layer 416, such as a pressure sensitive adhesive. A removable release layer 414 is provided to protect the adhesive layer until use, which should be made from or is provided with an easily removable surface to readily separate from the adhesive layer 416 when using the cover sheet. The cover sheet 400 can have an overall size or diameter to fit any injection site, ranging from about 0.5 inch in diameter and up. In an example, the cover sheet 400 is individually wrapped in a moisture and liquid impermeable over-wrap. A container or package of cover sheets can comprise a plurality of individually wrapped cover sheets, similar to a first-aid bandage package.

In an example, the impermeable layer 402 is made from a pliable thermoplastic or polymer material that can flex to follow the shapes or contours of the skin. Examples of thermoplastics and polymers useful for the backing layer are polyolefin, polyester, polyethylene, polyethylene vinyl acetate block copolymers, polyurethane, polyvinyl alcohol, polyvinylidene, polyvinylidene chloride, polyamide, ethylene-vinylacetate copolymer, ethylene-ethylacrylate copolymer, and polypropylene. The backing layer can also comprise laminates of one or more of the foregoing polymers. In an example, the impermeable layer 402 has a thickness of about one thousandths to about four thousandths of an inch.

In an example, the impermeable layer 402 of the cover sheet 400 has adhesive applied to the inner surface edge 410 adjacent the perimeter 408, but not to the central portion of the inner surface 406. In other words, the inner surface has a surface area without any adhesive and is surrounded by a ring of adhesive. With reference to FIG. 12 in addition to FIG. 10, a bottom view of the cover sheet 400 taken along line A-A is shown, without the release layer 414. As shown, the adhesive layer 416 forms a ring 420 around a central portion 424 of the inner surface 406 of the impermeable layer 402. The ring 420 can embody any number of shapes. The shapes can be round, oval, square, polygonal, irregular, etc. In an example, the ring of adhesive generally matches the shape of the cover sheet. In other examples, the shape of the cover sheet and the ring of adhesive can have shapes that differ from one another. For example, the impermeable layer 402 can have a square or rectangular shape while the ring 420 of adhesive can have an oval or circular shape.

When the cover sheet 400 of FIG. 10 is applied over an injection site, such as following an injection using the needleless injector 10 of FIGS. 1-5, the cover sheet 400, with the release layer 414 removed and the inner surface 406 facing the skin, is applied so that the adhesive ring 420 wraps or circles around the injection site and the injection site is retained within the inside perimeter of the ring 420. As further discussed below, any residual medicinal fluid not delivered subcutaneously by the needleless injector can be captured under the cover sheet and within the adhesive ring 420 to be deposited transdermally by the cover sheet through the puncture formed by the needleless injector. Thus, aspects of the present disclosure is understood to include a combination needleless injector and cover sheet usable for delivering residual medicinal or therapeutic fluid through the skin after formation or production of at least one micro-pore or microstructure, such as a micro-puncture or small puncture formed by a needleless injector.

The adhesive 416 can be a pressure sensitive adhesive, which is understood as a viscoelastic material that remains tacky and that can adhere to the skin with the application of pressure, from a very slight or light pressure to any higher pressure. Typically, the adhesive can be made from a polymer based material mixed with plasticizers, tackifiers or other additives. Suitable pressure sensitive adhesive includes the polyacrylate adhesives and acrylic adhesives.

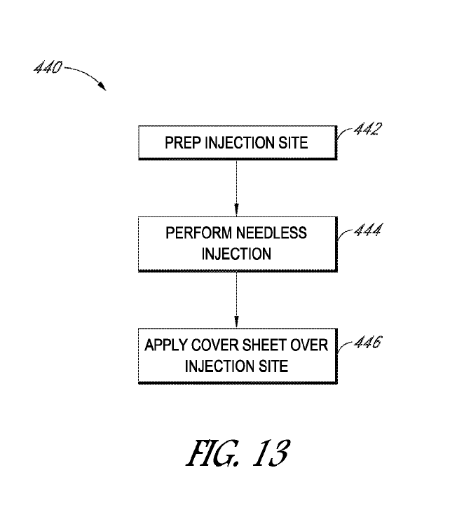

With reference now to FIG. 11, an alternative cover sheet 400 is shown. As shown, the alternative cover sheet 400 comprises a outer impermeable layer 402 similar to that of FIG. 10, an inner layer 424, an adhesive layer 416 similar to that of FIG. 10, and a release layer 414 similar to that of FIG. 10. In an example, the inner layer 424 can comprise an agent carrying layer 424. For example, the agent carrying layer can be an inner adhesive layer comprising a therapeutically effective amount of drug. Suitable transdermal drug delivery devices include gelled or liquid reservoirs, such as in U.S. Pat. No. 4,834,979; devices containing matrix reservoirs attached to the skin by an adjacent adhesive layer, such as in U.S. Pat. No. 6,004,578 (Lee, et al.); and devices containing pressure-sensitive adhesive reservoirs, such as in U.S. Pat. Nos. 6,365,178, 6,024,976, and 6,149,935. These devices are known as “drug-in-adhesive” patches and the disclosures of each of which are expressly incorporated herein by reference. In some examples, the drug carried by the inner layer 424 is hyaluronic acid. In other examples, the inner layer can embody an alginate dressing.

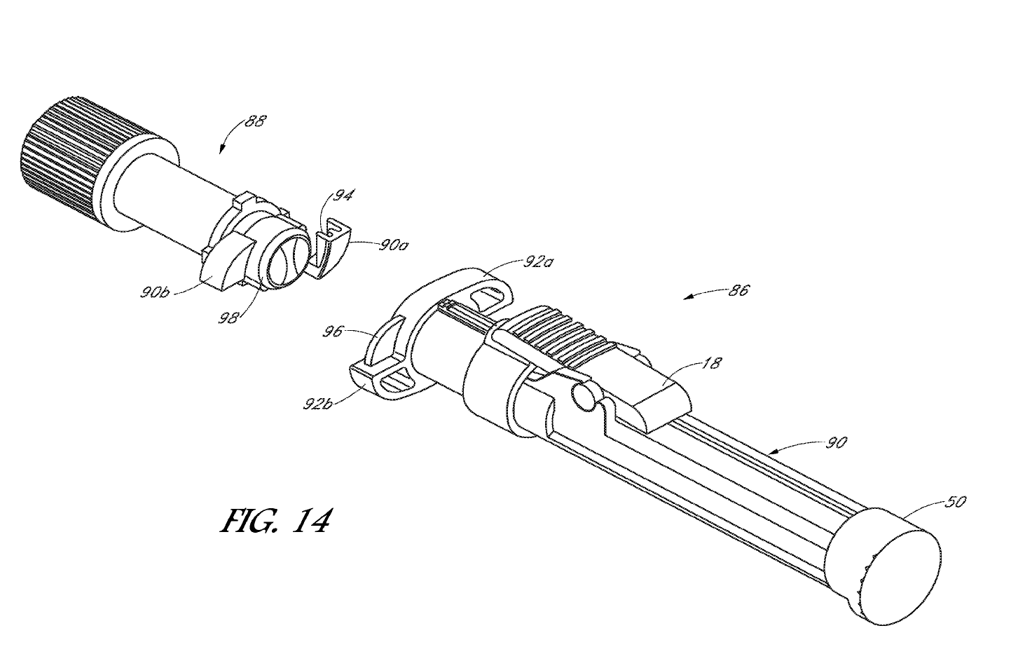

With reference now to FIG. 13, a process flow diagram 440 depicting a method of using the combination needleless injector and cover sheet of the present disclosure is shown. In an example, the method 440 comprises prepping an injection site 442, such as by wiping the skin or injection site with an alcohol wipe or an antiseptic wipe or towelette. Using a needleless injector, such as the needleless injector of FIGS. 1-5, a therapeutically effective amount of fluid is delivered subcutaneously at the prepped injection site, at step 444. The needleless injector may be filled with a therapeutically effective amount of fluid and used as discussed elsewhere herein. At step 446, the method further comprises applying a cover sheet 400 of the present disclosure over the injection site. In an example, an individually wrapped cover sheet 400 is first removed from its packaging. Then a release layer is removed from the cover sheet. The cover sheet is then applied over the injection with the adhesive ring circumscribing the micro-structure formed by the needleless injector. The cover sheet can help any residual or remaining quantity of liquid not delivered by the needleless injector absorb or penetrate the skin, such as through the micro-pore or micro-structure formed by the needleless injector.

Following the injection, the drug permeability of the skin at the injected area has been modified or induced by the high pressure stream expelled from the needle-free device. Thus, any residual drug that has not entered the skin can readily absorb at the injection site. The cover sheet 400 is configured to be placed over the injection site to form a boundary layer around any residual medicine and to facilitate absorption of the remaining or residual therapeutically effective fluid on the exterior of the skin to enter the skin. The ring of adhesive can help secure the cover sheet to the skin and surround the residual therapeutically active ingredient.

In some examples, the injection site can comprise more than one passage or hole created by the needleless injector. For example, the ampule on the needless injector can have more than one discharge nozzles, such as two or more discharge nozzles to create two or more micro-pores or micro-structures. In other examples, two different injections can be performed using two different needleless injectors each with an ampule with only one discharge nozzle. In still other instances, the same drive end 12 can be reset for use with two different ampules to perform two different needleless injections. The cover sheet 400 of the present disclosure can then be placed over the injection sites to surround two or more micro-pores or structures after the injection(s) to facilitate absorption of any residual liquid or medicine located above the skin to enter the skin through the micro-pores or micro-structures formed by one or more needless injectors of the present disclosure.

After the injection, according to the rise of the permeability, the barrier function of the skin can be expected to decrease against the infectious agents, such as bacteria of the skin. Thus, the sterilized over-lapping sheet or cover sheet of the present disclosure can protect from such risk during the skin recovery.

The over-lapping sheet or cover sheet should be easily or readily removable from the skin after about 24 hours. In some examples, the cover sheet can be removed much sooner than 24 hours, such as 30 minutes, 45 minutes, or 1 hour after application. Other elapsed times are contemplated.

With reference now to FIG. 14, an alternative needleless injector 86 is shown with the discharge end 88 separated or spaced from the drive end 90. In one example the needleless injector 86 is substantially the same as the needleless injector 10 of FIG. 1 except for the engagement mechanism between the discharge end 88 and the drive end 90. The engagement mechanism may be understood as a quick connect engagement mechanism to enable quick mounting of the discharge end 88 onto the drive end 90. As shown, the discharge end 88 comprises a pair of engagement wings 90a, 90b for mating engagement with the pair of corresponding mating wings 92a, 92b on the drive end 90. The engagement wings 90a, 90b on the discharge end each comprises a ramp 94 for sliding engagement with a tapered fin 96 formed on the corresponding mating wings 92a, 92b of the drive end 90. The ramp 94 and the tapered fin 96 act like a cam and axially load the two surfaces when the proximal end 98 of the discharge end 88 is placed into the drive end 90 and rotated to engage the engagement wings 90a, 90b with the mating wings 92a, 92b. Note that the discharge end 88 is shown without a plunger to better show the engagement mechanism. In practice, a plunger (similar to “70” in FIG. 2) would be disposed inside the discharge end 88 and partially extends outwardly of the proximal end 98. Other alternatives may be used to engage the discharge end 88 with the drive end 90. For example, notches and/or other matching features may be incorporated on the discharge end 88 and the drive end 90 to ensure compatibility before they can be connected.

In view of the foregoing, the present disclosure is understood to include a discharge end, such as an ampule, comprising a quick connect engagement mechanism. The present disclosure is further understood to include a pair of spaced apart engagement wings disposed about a proximal opening of the ampule for engaging with a pair of mating wings. In another embodiment, three spaced apart engagement wings are provided, which are evenly spaced about the proximal opening of the ampule. In another example, an injector end comprising a mating engagement mechanism is configured to quickly engage the engagement mechanism of the discharge end. In a particular example, two mating wings are provided at a distal end of the elongated body of the drive end for engaging spaced apart engagement wings on the discharge end. Other quick connect engagement mechanisms are contemplated, such as threads, detents, tongue and groove combination, etc.

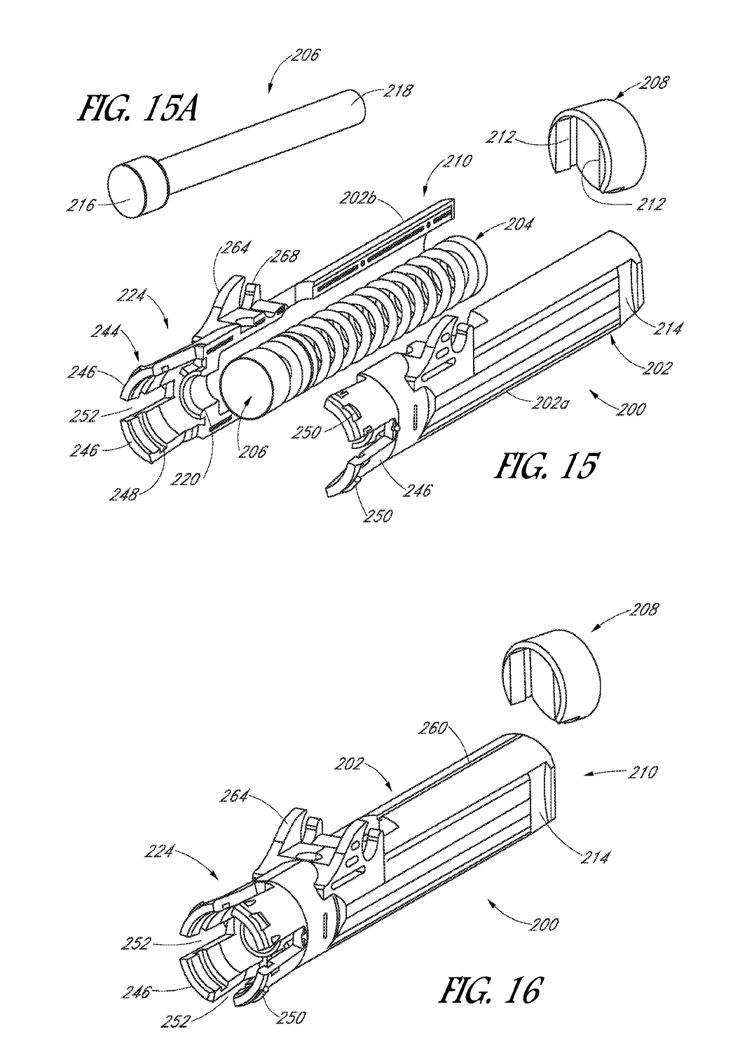

FIG. 15 is a schematic exploded perspective view of a drive end 200 of an alternative needleless injector device or needleless injector assembly provided in accordance with further aspects of the present system, device and method. Broadly speaking, the drive end 200, which may also be referred to as an injection driver, injector, power end, or spring end, comprises a multi-part housing 202, a motive force device 204, such as a metallic helical coil spring, a piston 206, and an end cap 208 for closing the proximal end 210 of the drive end 200. When assembled, internal engagement flanges 212 on the end cap 208 engages the external channel 214 on each of the two housing halves 202a, 202b, as discussed above. The two housing halves 202a, 202b after placing the motive force device 204 and the piston therebetween, may be glued, welded, latched, or a combination thereof together. FIG. 15A shows a perspective view of the piston 206, which comprises a piston head 216 and a piston stem 218 having a stem length that can vary and having a shoulder located therebetween.

As clearly shown on the housing body 202b of FIG. 15, a piston stopper 220 is formed near the distal end 224 of the housing. The piston stopper 220 defines an annular bore to permit cocking of the piston 206, such as by allowing a cocking tool to reach through the annular bore to compress the motive force device 204. After the motive force device 204 is compressed, a trigger finger 240 on the trigger 242 (FIG. 3) is positioned distally of the piston head 216 to hold the motive force device in the compresses position, such as holding the distal face of the piston head.

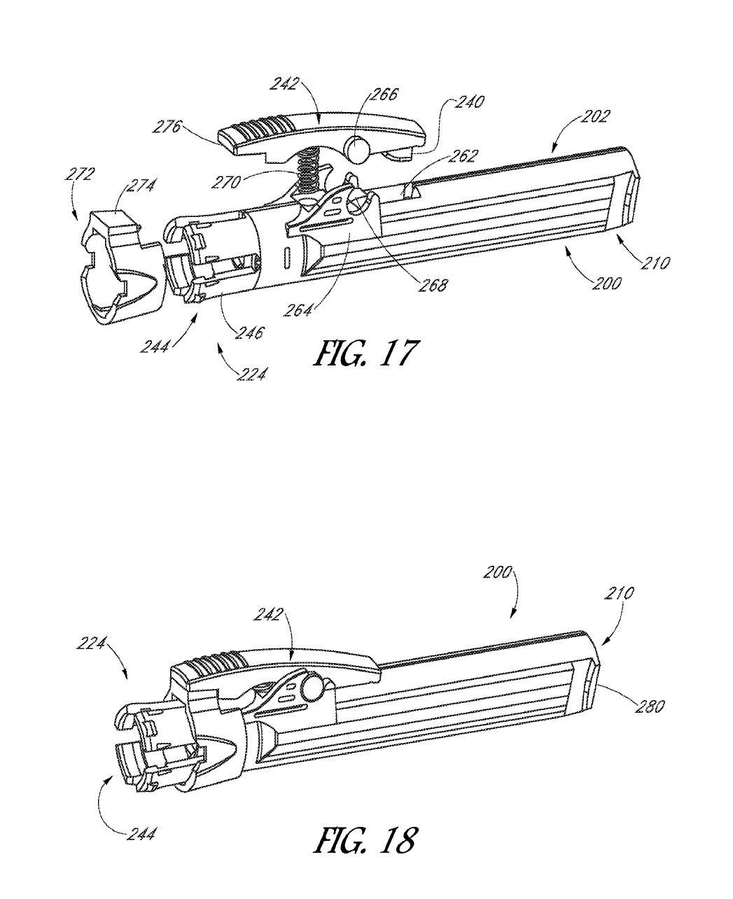

A connection mechanism 244 is formed at the distal end 224 of the drive end 200. As shown, the connection mechanism 244 comprises spaced apart latching tines 246 each with internal gripping ridges or fingers 248 for gripping a discharge end component, such as an ampule. The fingers are located interiorly on the latching tines 246 and ramped projections 250 are located exteriorly to enable a locking ring (FIG. 17, 272) to ride thereover to close the tines 246 over the discharge end component, such as an ampule, as further discussed below. The tines 246 are molded with an outward bias so that the locking ring is needed to force the tines inward to close against the discharge end component. A gap 252 is provided between two adjacent tines 246 to receive flanged sections of the discharge end component of ampule for alignment purposes. In other embodiments, the channels are reduced or omitted as the ampule can be provided as a round structure to eliminate alignment issue.

FIG. 16 is a perspective view of the drive end 200 assembled together and the seam 260 either welded or glued. The end cap 208 can now slide over the proximal end 210 with the engagement flanges 212 sliding into the channels 214 located on either side of the housing 202 at the proximal end. The drive end 200 is now ready to receive a trigger and a closing ring, as shown with reference to FIGS. 17 and 18, discussed below.

FIG. 17 shows a trigger 242 comprising a trigger finger 240 for projecting into the trigger hold slot 262 to hold the piston 206 and the motive force device 204 in the cocked or ready to use positioned. The trigger 242 is assembled to the cradle 264 by pushing the two pivot pins 266 on either side of the trigger into the slotted receptacles 268 of the two cradles 264, one on each housing half. The relative dimensions of the slotted receptacles hold the pins 266 in place after they are pushed therein. A rocker spring 270 is provided between the trigger 242 and the housing 202 to pivot the trigger about the two pivot pins 266. In on example, the rocker spring 270 may be omitted. In yet other examples, a compressible or elastic material, a leaf spring, or a V-spring may be used.

A closing ring 272 is shown for closing the tines 246. The closing ring 272 may be placed over the tines 246 by temporarily biasing the tines inwardly towards a longitudinal axis of the housing 202 so that the opening of the closing ring 272 can fit over and slide onto the housing. The closing ring 272 can further incorporate a trigger lock 274 that sits under the lock landing 276 on the trigger to provide a physical barrier against rocking or pivoting by the trigger, which prohibits the trigger from pivoting about the pivot pins. The closing ring 272 and the connection mechanism 244 can further incorporate notches and detents for alignment purposes and for thwarting the closing ring 272 from being displaced off of the connection mechanism.

FIG. 18 shows the drive end 200 in the fully assembled position except for the end cap 208. In one example, the housing 202 may be assembled as shown and then the piston 206 and the spring 204 slid into the housing 202 via the proximal opening 280 at the proximal end 210 of the housing. The end cap 208 is thereafter placed into engagement with the housing to close off the proximal opening 280. As discussed above, after an injection, the end cap 208 may be removed to enable separation of the metallic spring 204 from the other components of the injector end 200. This allows the drive end 200 and the discharge end component, such as an ampule, to be recycled without any metallic parts. Optionally, the trigger 242 may be removed from the housing 202 to enable removable of the rocker spring 270, if incorporated. If the rocker spring 270 is not incorporated, the drive end 202 may be recycled after the drive spring 204 is removed through the proximal opening 280.

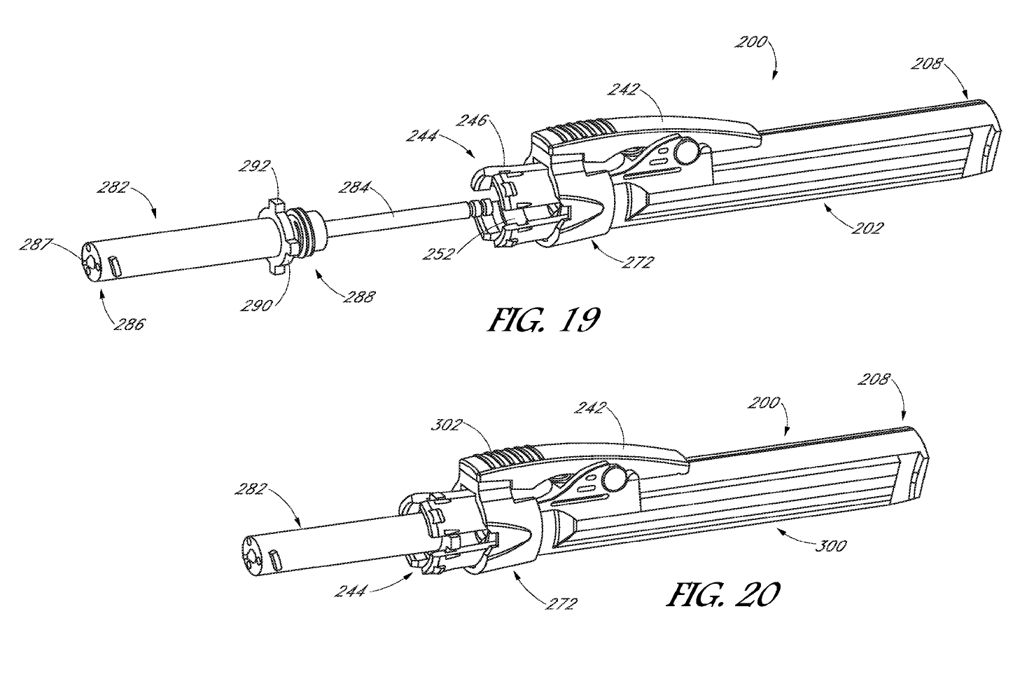

FIG. 19 shows an ampule 282 with a plunger 284 positioned for mounting onto the drive end 200. The ampule may be made from a cyclo-olefin-copolymer material (COC). As shown the ampule 282 comprises a discharge end 286 comprising a discharge nozzle 287 and a mounting end 288 comprising a mounting flange 290 and a plurality of flange projections 292. The flange projections 292 are sized and shaped to slide between the gaps 252 located between the plurality of tines 246 of the connection mechanism 244. The flange 290 is sized and shaped to sit within the gripping ridges 248 formed interiorly of the tines. Distal movement of the closing ring 272 once the mounting end 288 is placed into the connection mechanism 244 can force the tines 246 to close down on the flange and secure the ampule 282 to the drive end 200.

FIG. 20 shows the ampule 282 attached to the drive end 200 to form a needleless injector device or assembly 300 capable of delivering a dosage of medicinal fluid subcutaneously when the closing ring 272 is moved distally out from under the trigger 242 and the trigger is pressed at the triggering end 302. The assembly 300 can deliver a dosage subcutaneously without a needle.

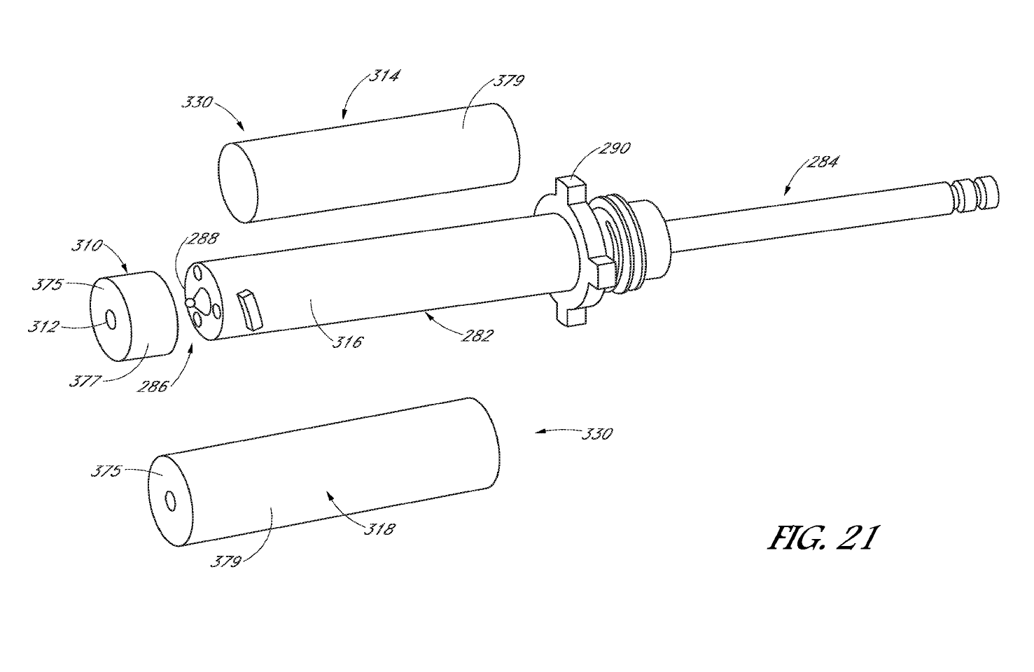

FIG. 21 shows an ampule 282 with different optional outer sleeves or outer layers. In one example, a discharge end sleeve 310 may be mounted over the discharge end 286 of the ampule 282. The discharge end sleeve 310 may be made from a transparent or semi-translucent elastomeric or rubber material and placed over the discharge end 286 to cushion the contact between the ampule and the recipient of the fluid to be delivered. The discharge and sleeve 310 has an end wall 375 and a skirt section 377. A distal opening 312 is provided on the end wall 375 of the discharge end sleeve 310 to provide the needed opening for the discharge nozzle 288.

Also shown in FIG. 21 is a body sleeve 314 comprising an elongated body section 379 for placement over the body 316 of the ampule 282. The body sleeve 314, like the discharge end sleeve 310, is preferably made from a transparent or semi-translucent elastomeric or rubber material to enable viewing the inside fluid holding space of the ampule, such as to visually ascertain the medicinal level inside the ampule. In one example, both the discharge end sleeve 310 and the body sleeve 314 are placed over the ampule 282. The combination discharge end sleeve and the body sleeve may be referred to as an outer layer, generically referred to as outer layer 330. For example, the discharge end component 282 may be made from a rigid plastic material and having an outer layer 330 placed thereover or thereon. The outer layer 330 is made from a different material than the discharge end component 282. In some embodiments, the outer layer 330 is made or formed from two separate pieces while in other embodiments from a single piece, as further discussed below. The body sleeve 314 of the outer layer 330 should extend more than half the length of the ampule. In a particular example, the length of the body sleeve should extend to the mounting flange 290, such as contact with or nearly contact with the mounting flange. In other examples, the length of the outer layer 330 can extend to around the half-way point of the length of the ampule and up to the mounting flange 290. If there is no mounting flange, then up to about the interface of the ampule and the injector end. It is believed that the impulse of force from the initial release of the motive force or spring 204 and subsequent rapid movement of the plunger into the ampule, such as upon releasing the spring of FIG. 9, can be dampened by the use of the outer layer 330. Thus, the outer layer 330 should have a tight formfitting configuration around the body 316 of the ampule to dampen some of the initial shock experienced by the ampule upon releasing the spring.

FIG. 21 also shows the outer layer 330 as a one-piece sleeve 318 for mounting over the ampule 282. The one-piece sleeve 318 can be thought of as a combination of the discharge end sleeve 310 and the body sleeve 314. The sleeve 318 has an end wall 375 and an elongated body section 379. The sleeve 318 is preferably sized and shaped to firmly wrap over the outside surface of the ampule but not so rigid or tight so as to deter or make assembling or mounting the sleeve over the ampule difficult. In some examples, the outer layer 330 only has an elongated body section without a distal wall, such as an open cylinder.

FIG. 22 shows an ampule 282 having an outer layer 330 mounted thereon, which comprises a discharge end sleeve 310 and a body sleeve 314. FIG. 23 shows an ampule 282 having an outer layer 330 mounted thereon, the outer layer comprising a one-piece sleeve 318. The ampules of FIGS. 22 and 23 may be mounted for use with any one of the injector ends discussed elsewhere herein.

FIG. 24 shows another embodiment of an outer layer 330. As shown, a shrink wrap bag 332 is provided for mounting over an ampule. The shrink wrap bag 330 has an elongated body section 338 with a length, an inside diameter, and a distal opening 334 formed on a distal end, distal wall, or end wall 336 of the bag. The inside diameter of the shrink wrap bag 330 is sized sufficiently larger than the outside diameter of an ampule for which the outer layer is to be used with and the distal opening 334 has a perimeter that is sufficiently larger than the nozzle on the ampule so as not to obstruct fluid flow passing through the nozzle. In use, the shrink wrap bag 332 may be slid over the body of an ampule and then subjected to heat so that the outer layer 330 shrinks to form a tight fitting around the ampule. Heat may be provided from electricity or from a gas source and may be part of a heated tunnel or an oven. Heated lamps may also optionally be used to shrink the outer layer 330.

In some examples, the shrink wrap bag 332 may be made from a polymer material. Preferred polymers used for the shrink wrap bag 332 include polyolefin, polyvinyl chloride (PVC), and polyethylene. The materials may be cross-linked or non-cross-linked. The shrink wrap bag 332 may be formed to shrink in one direction (i.e., unidirectional or mono-directional) or in two directions (bidirectional). For example, the shrink wrap bag 332 of FIG. 24 may be configured to only shrink in diameter but not length. In other examples, the shrink wrap bag may be configured to shrink both in diameter and along its length. The final length should extend at least to a half-way point of the length of the ampule and up to the mounting flange on the ampule, if any. If there is no mounting flange, then up to about the interface of the ampule and the injector end.

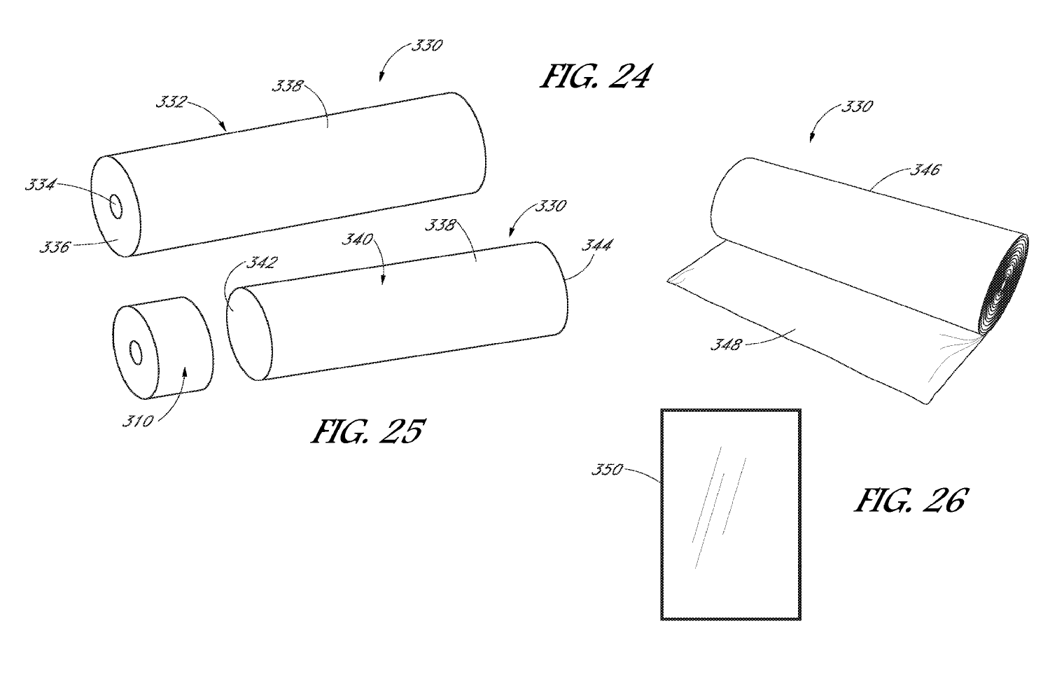

FIG. 25 shows another embodiment of an outer layer 330. As shown, the outer layer comprises a shrink wrap open cylindrical section 340 and a discharge end sleeve 310, similar to the end sleeve 310 of FIG. 21. The open cylindrical section 340 has an elongated body section 338 with two open ends 342, 344. The elongated body section 338 is sized and shaped to slide over an ampule and the first open end 342 is sized and shaped to let the discharge end of the ampule left exposed. Other characteristics of the layer 340 are the same as that of the embodiment of FIG. 24. Once the layer 340 is heated and wrap tightly or snuggly around an ampule, the discharge end sleeve 310 may be placed over the end of the ampule. Thus, when the outer layer 330 of FIG. 25 is mounted over an ampule, the combination has at least three different materials—one being from the ampule itself, the second from the shrink wrap material, and the third being from the discharge end sleeve.

FIG. 26 shows yet another embodiment of an outer layer 330, which is shown as a roll 346 of shrink wrap sheet or layer 348. The layer 348 may be cut down to a working size sheet 350, applied over an ampule, and then subjected to heat to set over the ampule. The working size sheet 350 may be rectangular in shape having a length and a width. Alternatively, the roll 346 may be made as a stretch wrap layer or material. Stretch wrap materials are highly stretchable plastic films. The elastic recovery of each film keeps the item that the film is applied against tightly bound. The layer 348 may be cut down to size, stretched and applied over an ampule to keep a tight fit over the ampule.

In use with an ampule, the outer layers 330 of FIGS. 24-26 look similar to that shown in FIGS. 22 and 23. The ampules with the outer layers 330 of FIGS. 24-26 may be used with any of the injector ends discussed elsewhere herein.

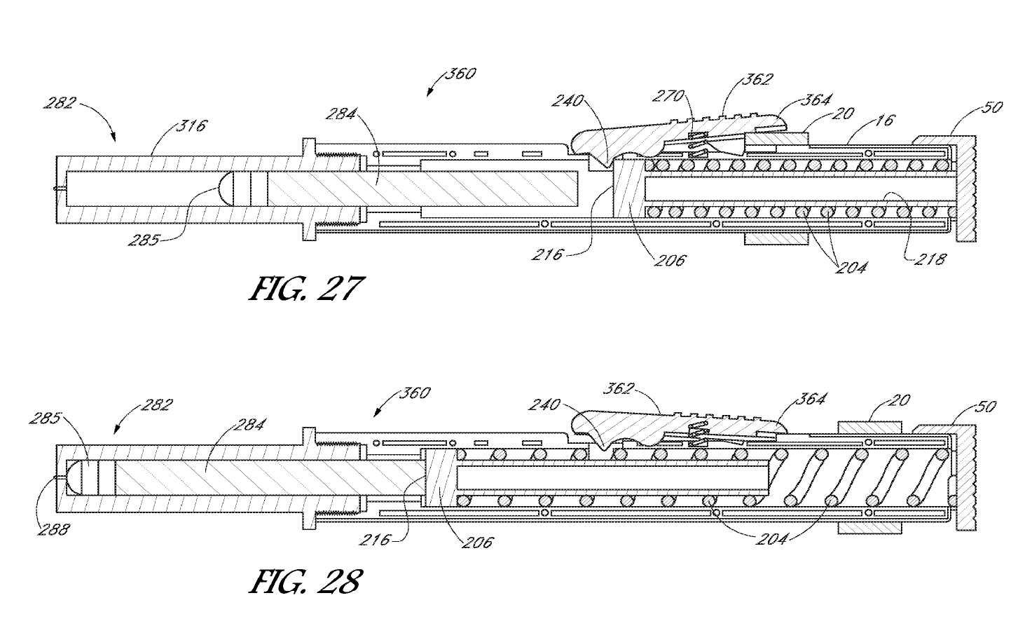

With reference now to FIG. 27, a semi-schematic cross-sectional side view of another needleless injector assembly 360 provided in accordance with aspects of the present disclosure is shown. The assembly 360 comprises an injector end component 362 and a discharge end component 282, which are similar or comparable to components previously discussed elsewhere herein. Thus, the assembly is understood to include an ampule body 316, a plunger 284 with a plunger tip 285, a piston 206 with a piston head 216 and a piston stem 218, a spring or motive force 204 for propelling the piston head 216 into the plunger 284, a safety lock 20, and an end cap 50 for closing off the proximal opening of the elongated housing body 16. The cap 50 is removable following use to enable removable of the internal components, as previously discussed.

The assembly 360 is shown in a ready to use position in FIG. 27 with the latch pin or trigger finger 240 of the trigger 362 in contact with the piston head 216 to hold the spring 204 in a compressed position. However, unlike the embodiments of FIGS. 1, 3, 5, 14, and 18, the trigger 362 has a push end 364 that is pointed in the proximal direction, towards the end cap 50. In other embodiments, the push end of the trigger points distally towards the ampule. This may be arranged by changing the location of the cradle for holding the pivot pin that allows the trigger 362 to rotate to release the spring. The assembly 360 of FIG. 27 is ideally suited for use in applications that require the user to reach into a closed or confined space. For these applications, the user can still have access to the push end 364 of the trigger 362 to perform an injection. For example, when used in a dental application, such as to perform a local anesthesia or lidocaine injection, the direction of the push end 364 allows the clinician to insert the ampule into the mouth to inject the gum near the molars while still allowing for easy reach or access to the trigger to release the spring and perform the injection.

In practice, the size of the assembly 360 may be modified for different applications. For example, for dental applications or for small dosage applications, the size of the discharge end and injector end may be reduced. Furthermore, while the assembly 360 is described for use with confined space, it is not so limited and may be used in any wide open space application where subcutaneous delivery of medication is desired.

FIG. 28 is a cross-sectional side view of the injector assembly 360 of FIG. 27 following release of the spring 204. As shown, the safety lock 20 has been moved proximally to the right of FIG. 28, which provides space for the trigger 362 to be depressed at the push and 364. This causes the latch pin or trigger finger 242 to separate from the piston head 216. This in turn allows the spring 204 to expand and push the piston 206 forward to then push the plunger 284 forward to push medication or fluids inside the ampule out of the discharge nozzle 288 of the ampule 282.

Methods of making or forming components of the injector assemblies and cover sheets discussed herein as wells as completed injector assemblies discussed herein are understood to be within the spirit and scope of the present disclosure. Methods of using components of the injector assemblies and cover sheets discussed herein as wells as using the completed injector assemblies discussed herein are also understood to be within the spirit and scope of the present disclosure. Still furthermore, the present needleless injector assemblies are also ideally suited for being pre-filled and packaged inside a blister pack or package so that a user simply has to pry open the package, placed the discharge end of the nozzle against the skin, and squeeze the trigger to deliver a dosage of fluid subcutaneously. In another example, only the ampule end is pre-filled and packaged inside a blister pack.

Although limited embodiments of the needleless injector assemblies and their components have been specifically described and illustrated herein, many modifications and variations will be apparent to those skilled in the art. For example, the engagement between the ampule and the drive end can be other than as shown, etc. Different materials may also be used to form the various components described herein. Furthermore, it is understood and contemplated that features specifically discussed for one needleless injector embodiment may be adopted for inclusion with another embodiment provided the functions are compatible. Accordingly, it is to be understood that the needleless injector assemblies and their components constructed according to principles of the disclosed device, system, and method may be embodied other than as specifically described herein. The disclosure is also defined in the following claims.

What is claimed is:

1. A method of delivering a therapeutic amount of fluid subcutaneously without a needle comprising:
depressing a trigger on a needleless injector device comprising an ampule an injection driver at an injection site, said ampule comprising a nozzle at a discharge end and a slidable plunger and said injection driver comprising a spring and a piston;
applying a cover sheet over the injection site, said cover sheet comprising a liquid impermeable layer comprising an outer surface side and an inner surface side that faces the injection site, said cover sheet comprising a continuous adhesive ring located on the inner surface side of the liquid impermeable layer; and
wherein said adhesive ring defines a region external of the adhesive ring without adhesive and a region internal of the adhesive ring without adhesive.

2. The method of claim 1, further comprising prepping the injection site by wiping with an alcohol wipe or an antiseptic wipe prior to depressing the trigger.

3. The method of claim 1, wherein the cover sheet is removed from a water impermeable overwrap prior to applying the cover sheet over the injection site.

4. The method of claim 3, wherein the cover sheet is removed from a bulk package comprising a plurality of individually wrapped cover sheets.

5. The method of claim 3, wherein the cover sheet has a shape that is round, oval, square, or rectangular.

6. The method of claim 5, wherein the adhesive ring has a shape that is round, oval, square, or rectangular.

7. The method of claim 5, further comprising an inner layer adhered to the impermeable layer and the adhesive ring adhered to the inner layer.

8. The method of claim 5, wherein the therapeutic amount of fluid is hyaluronic acid.

9. The method of claim 5, wherein the inner layer comprises an alginate dressing.

10. A combination needleless injector device and cover sheet comprising:
an ampule comprising a body having a discharge end with at least one nozzle and a plunger slidably disposed inside the body;
an injection driver comprising an elongated body comprising a distal end comprising a distal opening having the ampule attached thereto and an interior surface defining an interior cavity having a spring and a piston located therein;
a trigger pivotably disposed about an exterior of the elongated body comprising a latch pin holding the piston against an expansion force of the spring, said piston comprising a shoulder in contact with the spring;
a cover sheet wrapped inside a liquid impermeable overwrap, said cover sheet comprising a liquid impermeable layer comprising an outer surface side, an inner surface side, and a continuous adhesive ring located on the inner surface side of the liquid impermeable layer; and
wherein said adhesive ring defines a region external of the adhesive ring without adhesive and a region internal of the adhesive ring without adhesive.

11. The combination of claim 10, further comprising an outer layer disposed around the ampule.

12. The combination of claim 11, wherein the outer layer comprises an end wall comprising an opening and wherein the opening on the end wall is disposed around the at least one nozzle on the ampule.

13. The combination of claim 10, wherein the cover sheet is located in a bulk package comprising a plurality of individually wrapped cover sheets.

14. The combination of claim 14, further comprising an inner layer adhered to the impermeable layer and the adhesive ring adhered to the inner layer.

15. A cover sheet for use with a needleless injector device comprising:
a liquid impermeable layer with adhesive located inside a liquid impermeable overwrap, said liquid impermeable layer comprising an outer surface side, an inner surface side, and a continuous adhesive ring located on the inner surface side of the impermeable layer; and
wherein said adhesive ring defines a region external of the adhesive ring without adhesive and a region internal of the adhesive ring without adhesive.
